(19) United States
(12) Patent Application Publication
(10) Pub. No.: US 2019/0322767 A1
(43) Pub. Date: Oct. 24, 2019

(54) HETERODIMERIC ANTIGEN BINDING PROTEINS

(71) Applicant: INNATE PHARMA, MARSEILLE (FR)

(72) Inventor: LAURENT GAUTHIER, MARSEILLE (FR)

(21) Appl. No.: 16/472,233

(22) PCT Filed: Dec. 21, 2017
(86) PCT No.: PCT/EP2017/084056

§ 371 Date: Jun. 21, 2019

Related U.S. Application Data
(60) Provisional application No. 62/438,473, filed on Dec. 23, 2016.

(51) Int. Cl. C07K 16/46; C07K 16/28; C07K 16/30; A61P 35/00

(52) U.S. Cl. CPC C07K 16/468; A61K 2039/505; C07K 16/2827; C07K 16/30; C07K 16/2803; C07K 16/2887; C07K 16/2863; A61P 35/00; C07K 2317/732; C07K 2317/31; C07K 2317/72; C07K 2317/524; C07K 2317/526; C07K 2317/41; C07K 16/283

(57) ABSTRACT

Provided are heterodimeric proteins formed from dimerization between CH1 and CK domains and that bind a target antigen on a cell to be depleted. The proteins have advantages in production and in the treatment of disease, notably solid tumors or infectious disease.

### CROSS-REFERENCE TO RELATED APPLICATIONS

This application claims the benefit of U.S. Provisional Application No. 62/438,473 filed Dec. 23, 2016, which is incorporated herein by reference in its entirety; including any drawings and sequence listings.

### FIELD OF THE INVENTION

Binding proteins that bind and can be used to specifically direct effector cells to lyse a target cell of interest are provided. The proteins formats have utility in the treatment of disease.

### REFERENCE TO THE SEQUENCE LISTING

The present application is being filed along with a Sequence Listing in electronic format. The Sequence Listing is provided as a file entitled “BISP5 PCT_ST25 txt”, created Dec. 21, 2017, which is 178 KB in size. The information in the electronic format of the Sequence Listing is incorporated herein by reference in its entirety.

### BACKGROUND

Antibodies having greatest efficacy in eliciting CD16-mediated ADCC activity in vivo have generally been full-length antibodies, i.e. tetrameric proteins that are capable of bivalent binding to their target antigen on target cells. An extensive body of literature describes amino acid modifications (generally substitutions) in the Fc domain of antibodies, generally of IgG1 isotype, that increases binding to activating human Fc receptors such as CD16. These modified Fc domains provided increased potency in the ability to induce ADCC towards target cells. However, despite the existence of a variety of enhancement to Fc domains for depleting antibodies, there is therefore a need in the art for therapeutic proteins with improved potency in tissues, particularly for the treatment of cancers such as solid tumors, and that have attractive properties for industrial development.

### SUMMARY OF THE INVENTION

The present invention arises from the discovery of a functional Fc-protein format having high potency in mediating ADCC. The protein of the invention has comparable or better potency in ADCC assays compared to conventional full-length antibodies that bind their cellular targets bivalently (e.g., a conventional full length human IgG1 antibody having two antigen binding domains), yet is of small size and thereby can present advantages in pharmacology, particularly in addressing molecular targets found in tissues, e.g. extravascular tissues, solid tumors.

The protein can be advantageously prepared as a heteromultimeric (e.g. heterodimeric) antigen binding protein comprising an antigen binding domain and an Fc domain, wherein the protein binds an antigen of interest on a target cell to be depleted via a single antigen binding domain and wherein the protein binds immune effector cells solely via its Fc domain. In one embodiment the protein is prepared as a heterodimeric antigen binding protein comprising a single antigen binding domain comprised of one VH and one VL domain (a VH-VL pair), a CH1-CK pair (a CH1 domain and a CK domain, placed on different polypeptide chains adjacent to the VH or VL domain such that they undergo interchain CH1-CK dimerization) and a dimeric Fc domain (e.g. comprised of a pair of Fc domain polypeptides that bind to one another via CH3-CH3 association). Furthermore, in one embodiment of any protein herein, when the protein of the invention comprises a modified Fc domain to enhance affinity for human CD16, the protein can be characterized by comparable or better potency in ADCC assays compared to a full-length antibody that comprises the same modified Fc domain.

The heteromultimeric protein format described herein permits a wide range of antibody variable regions to be readily used as V_{H} and V_{L} pairs, without a need to identify or engineer V domain pairs that retain affinity in single chain arrangements. The protein format of the disclosure also has advantages in manufacturing by being adapted to standard recombinant production techniques and without the need for development of product-specific folding or purification techniques. While the new protein formats can be used to bind any desired antigens by incorporation the desired variable regions, advantageous examples are provided where monovalent proteins can bind to an antigen of interest on the surface of a target cell (e.g. a cell to be eliminated or depleted via ADCC).

The proteins will generally possess a single antigen binding domain (ABD), e.g., formed by immunoglobulin variable regions, e.g., a V_{H} domain and a complementary V_{L} domain, thereby displaying monovalent binding to a target antigen, and a dimeric Fc domain that binds the human activating receptor CD16A (FcγRIIIA). The dimeric Fc domain may optionally further any one or more of the other human Fcγ receptors, e.g., FcγRI (CD64), FcγRIIA (CD32A), FcγRIIB (CD32B), CD16B, as well as human FcRn. The Fc domain is preferably of human origin (a human Fc domain) and comprises N-linked glycosylation. Each V domain can be placed on a separate polypeptide chain and fused (directly or via intervening amino acid sequences) to a CH1 or CK constant domain, such that the one polypeptide chain bears the CH1 and another chain bears the CK domain and the two chain associate via CH1-CK heterodimerization and the VH and VL domain form the ABD. The proteins can thereby bind a target antigen of interest in monovalent fashion via the ABD (the protein comprises a single binding site for the antigen of interest), and the Fc domain binds to human CD16 (optionally in combination with further human FcγR proteins) as well as the human neonatal Fc receptor (FcRn).

In one embodiment, provided is a heterodimeric antigen binding protein (e.g., a monovalent antigen binding protein) comprising a single antigen binding domain (ABD) and a dimeric Fc domain that comprises N-linked glycosylation and binds the human activating receptor CD16A. The ABD binds in monovalent fashion to the antigen of interest (e.g., a predetermined antigen of interest). The protein can comprise two different polypeptide chains (the protein can be referred to as a heterodimer herein). Each of the polypeptide chains can comprise a heavy or light chain variable domain fused (e.g. at its C-terminus, optionally via intervening amino acid sequences such as flexible linker peptide) to a human CH1 or Cκ constant domain (a V−(CH1/Cκ) unit), and the CH1 or Cκ constant domain is fused (e.g. at its C-terminus, optionally via intervening amino acid sequences such as an immunoglobulin hinge sequence) to an Fc domain. The protein (or a polypeptide chain thereof) can be specified to be free of any further protein domain (or of any immunoglobulin variable domain) fused to the C-terminus or positioned C-terminal of the Fc domain. The two polypeptide chains will thereby undergo CH1-Cκ heterodimerization so as to be bound to one another by non-covalent bonds formed between respective CH1 and Cκ domains and optionally further by disulfide bonds. The Fc domains of the two chains will pair such that a dimeric Fc domain is formed. In one embodiment, the monovalent protein is smaller than a conventional human IgG antibody. In one embodiment, the monovalent protein is characterized by decreased freedom of motion between the ABD and Fc domain than the freedom of motion between an ABD and Fc domain in a conventional human IgG antibody.

In one embodiment, provided is an isolated or purified heterodimeric protein comprising a single binding site for an antigen of interest.

In one embodiment, a heterodimeric protein comprises two polypeptide chains each comprising a different V—(CH1/Cκ) unit, wherein the two chains further comprise an Fc domain fused to the C-terminus of the V—(CH1/Cκ) unit, whereby the chains are bound to one another by non-covalent bonds and optionally further by disulfide bonds between CH1 and Cκ domains, optionally, whereby the chains are further bound by non-covalent bonds between respective variable regions, CH1 and Cκ domains, and wherein the two chains are further bound by non-covalent bonds between CH3 domains of the respective Fc domains. The protein (or a polypeptide chain thereof) can be specified to be free of any further immunoglobulin variable domains.

In any embodiment herein, the protein (or one or both polypeptide chain thereof) can be specified to be free of any further protein domain (or of any immunoglobulin variable domain) fused to the C-terminus or positioned C-terminal of the Fc domain.

In any embodiment herein, the protein (or a polypeptide chain thereof) can be specified to have a single VH domain and a single VL domain.

The variable and constant regions are selected and configured such that each chain will preferentially associate with its desired complementary partner chain. The resulting multimeric protein will therefore be simple to produce using conventional production methods using recombinant host cells. The choice of which V_{H} or V_{L} to associate with a CH1 and Cκ in a unit is based on affinity between the units to be paired so as to drive the formation of the desired multimer. The resulting dimer will be bound by non-covalent bonds between complementary V_{H} and V_{L} domains, by non-covalent bonds between complementary CH1 and Cκ domains, and optionally disulfide bonding between complementary CH1 and Cκ domains (and/or optionally further disulfide bonds between complementary hinge domains). Additionally, by including an Fc domain, preferred chain pairing is further improved, as the two Fc-containing chains will be bound by non-covalent bonds between CH3 domains of the Fc domains.

In one embodiment, the Fc domain comprises N-linked glycosylation at residue N297 (Kabat EU numbering).

In one example, the protein has a domain arrangement:

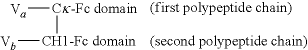

wherein Vₐ and V_{b} are each a V_{H} or V_{L} domain, and wherein one of Vₐ and V_{b} is a V_{H} domain and the other is a V_{L} domain. The Fc domains associate (e.g. via CH3 dimerization) to form a dimeric Fc region that is bound by CD16 (e.g. human CD16). Optionally, the CH1 and CK domains are each fused to the Fc domain via a hinge domain. Optionally, each Vₐ and V_{b} domains is fused to the respective Cκ or CH1 domain via a linker sequence, e.g. an amino acid sequence RTVA.

Optionally, in any embodiment, the Cκ domain is fused to the Fc domain via a linking amino acid sequence derived from a hinge domain, e.g. a native or modified hinge domain or fragment thereof. Optionally, in any embodiment, the CH1 domain is fused to the Fc domain via a hinge domain, e.g. a native or modified hinge domain. In one embodiment, each of the CH1 and CK domain is fused to a Fc domain via a hinge domain, e.g. a native or modified hinge domain. In one embodiment, interchain disulfide bonds are formed between hinge domains of the two polypeptide chains.

In any embodiment, a hinge domains may be a full hinge domain or a fragment of a hinge domain. In any embodiment, a CH1 domain may be a full CH1 domain or a fragment of a CH1 domain. In any embodiment, a Cκ domain may be a full Cκ domain or a fragment of a Cκ domain.

In one example, the protein has a domain arrangement:

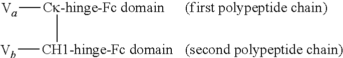

wherein Vₐ and V_{b} are each a V_{H} or V_{L} domain, and wherein one of Vₐ and V_{b} is a V_{H} domain and the other is a V_{L} domain.

In one example, the protein has a domain arrangement:

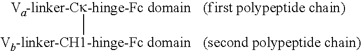

wherein Vₐ and V_{b} are each a V_{H} or V_{L} domain, and wherein one of Vₐ and V_{b} is a V_{H} domain and the other is a V_{L} domain.

In one embodiment, provided is a a heterodimeric protein comprising: (a) a first polypeptide chain comprising a variable domain fused at its C-terminus to a polypeptide chain comprising an amino acid sequence at least 60%, 70%, 80%, 90%, 95% or 99% identical to the sequence shown in SEQ ID NO: 12, and (b) a second polypeptide chain comprising a variable domain fused at its C-terminus to a polypeptide chain comprising an amino acid sequence at least 60%, 70%, 80%, 90%, 95% or 99% identical to the sequence shown in SEQ ID NO: 13, wherein one of the variable domains is a VH domain and the other is a VL domain.

The subject antigen binding proteins are well suited for use as depleting agents, e.g., that lead to the eliminating of target cells by immune effector cells (e.g. NK cells). The proteins are thus well suited for targeting antigens of interest expressed by target cells and for eliminating target cells, e.g., cells that are sought to be depleted such as tumor cells, cells in the tumor environment that contribute to tumor growth, or infected cells. Furthermore, in view of their monovalent binding, they may induce less down-modulation or intracellular internalization of the antigen of interest. Thus in one embodiment, the protein binds an antigen(s) known to be capable of undergoing down-modulation or internalization when bound by conventional antibodies (e.g. antibodies with human IgG1 Fc domains that retain CD16 binding).

In one embodiment, the target antigen or antigen of interest is a protein present in the tumor environment, e.g. a protein expressed by tumor cells or a soluble or cell-surface protein present in tumor tissue or tumor-adjacent tissue. In one embodiment, the target antigen or antigen of interest is a CD19, CD20, EGFR or PD-L1 protein. Optionally the monovalent binding protein binds PD-L1 neutralizes the inhibitory activity of PD-1, e.g., the monovalent binding protein binds PD-L1 and inhibits the interaction between PD-L1 and PD-1.

Provided also is a purified or homogenous composition, wherein at least 90%, 95% or 99% of the proteins in the composition are a dimeric polypeptide of the disclosure, e.g. proteins comprised of the two polypeptide chains and having the domain structure indicated herein.

In any embodiment, the ABD is comprised of one immunoglobulin heavy variable domain and one immunoglobulin light chain variable domain. An ABD represents a single antigen binding site and will bind in monovalent manner to its target. It will be appreciated however that where the ABD cross-reacts with more than one antigenic target it will be capable of binding via a single antigen binding site, but may bind to any one of a plurality of different antigens (including alleles or any differing forms of a particular antigen).

In one embodiment, the proteins have decreased freedom of motion of the antigen binding domains relative to the Fc domain (e.g. compared to the ABDs of a conventional human IgG antibody, e.g., a human IgG1 antibody).

Optionally in any embodiment, fusions or linkages on the same polypeptide chain between different domains (e.g., between V domains and CH1 or Cκ domains, between CH1 or Cκ domains and Fc domains) may occur via intervening amino acid sequences, for example via a hinge region or linker peptide. Any domains herein can optionally be fused or connected to an adjacent domain on a polypeptide chain by a linker. A linker can be a polypeptide linker, for example peptide linkers comprising a length of at least 5 residues, at least 10 residues, at least 15 residues, at least 20 residues, at least 25 residues, at least 30 residues or more. In other embodiments, the linkers comprises a length of between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-30 residues, between 10-24 residues, between 10-26 residues, between 10-30 residues, or between 10-50 residues. Optionally a linker comprises an amino acid sequence derived from an antibody constant region, e.g., an N-terminal CH1 or hinge sequence. Optionally, a linker (e.g., placed between a variable domain and a CH1 or CK constant domain) comprises the amino acid sequence RTVA.

Optionally in any embodiment, an antigen binding domain comprises the hypervariable regions, optionally the heavy and light chain CDRs, of an antibody, optionally one immunoglobulin V_{H} and one immunoglobulin V_{L}. Optionally in any embodiment, a variable domain comprises framework residues from a human framework region, e.g., a variable domain comprises 1, 2 or 3 CDRs of human or non-human origin and framework residues of human origin.

Optionally in any embodiment, the antigen of interest is a cancer antigen, viral antigen or bacterial antigen, or a polypeptide expressed on the surface of an immune cell.

In one aspect of any of the embodiments of the invention, the protein has a Kd for binding (monovalent) to an antigen of interest of less than 10⁻⁷ M, preferably less than 10⁻⁸ M, or preferably less than 10⁻⁹ M, as assessed by surface plasmon resonance (e.g., according to methods disclosed in in PCT application number PCT/EP2016/064537, filed 23 Jun. 2016 (Innate Pharma).

In one embodiment of any of the proteins herein, the monovalent protein is capable of directing CD16-expressing effector cells (e.g. a T cell, an NK cell) to lyse a target cell expressing the antigen of interest (e.g. a cancer cell, a virally infected cell, a bacterial cell, a pro-inflammatory cell, etc.).

In one embodiment of any of the proteins herein, the monovalent antigen binding protein comprises a dimeric Fc domain capable of binding to human CD16, and the protein is capable of directing effector cells (e.g. a T cell, an NK cell) that express human CD16 to lyse a target cell expressing one or more of the antigens of interest (e.g. a cancer cell). In one embodiment, the protein causes lysis of the target cell at least in part by CD16-mediated antibody-dependent cell-mediated cytotoxicity (“ADCC”).

In one aspect of any of the embodiments herein, provided is a recombinant nucleic acid encoding a first polypeptide chain, and/or a second polypeptide chain (and optionally further a third polypeptide chain) of any of the proteins of the disclosure. In one aspect of any of the embodiments herein, provided is a recombinant host cell comprising a nucleic acid encoding a first polypeptide chain, and/or a second polypeptide chain (and optionally further a third polypeptide chain) of any of the proteins of the disclosure, optionally wherein the host cell produces a protein of the disclosure with a yield (final productivity, following purification) of at least 1, 2, 3 or 4 mg/L. Also provided is a kit or set of nucleic acids comprising a recombinant nucleic acid encoding a first polypeptide chain of the disclosure, and a recombinant nucleic acid encoding a second polypeptide chain of the disclosure (and, optionally, a recombinant nucleic acid encoding a third polypeptide chain of the disclosure). Also provided are methods of making heterodimeric and heterotrimeric proteins of the disclosure.

In one embodiment, the invention provides methods of making a heterodimeric protein, comprising:

a) providing a first nucleic acid encoding a first polypeptide chain described herein;

b) providing a second nucleic acid encoding a second polypeptide chain described herein; and

c) expressing said first and second nucleic acids in a host cell to produce a heterodimeric protein comprising said first and second polypeptide chains, respectively; and

recovering a heterodimeric protein. Optionally, the heterodimeric protein produced represents at least 20%, 25% or 30% of the total monovalent proteins obtained prior to purification. Optionally step (d) comprises loading the protein produced onto an affinity purification support, optionally an affinity exchange column, optionally a Protein-A support or column, and collecting the heterodimeric protein; and/or loading the protein produced (e.g., the protein collected following loading onto an affinity exchange or Protein A column) onto an ion exchange column; and collecting the heterodimeric fraction.

By virtue of their ability to be produced in standard cell lines and standardized methods with high yields, unlike small protein formats such as single chain formats, the proteins of the disclosure also provide a convenient tool for screening for the most effective variable regions to incorporate into a monovalent binding protein. In one aspect, the present disclosure provides a method for identifying or evaluating candidate variable regions for use in a heterodimeric protein, comprising the steps of:

a) providing a plurality of nucleic acid pairs, wherein each pair includes one nucleic acid encoding a heavy chain candidate variable region and one nucleic acid encoding a light chain candidate variable region, for each of a plurality of heavy and light chain variable region pairs (e.g., obtained from different antibodies binding the same or different antigen(s) of interest);

b) for each of the plurality nucleic acid pairs, making a heterodimeric protein by:

(i) producing a first nucleic acid encoding providing a first nucleic acid encoding a first polypeptide chain described herein;
(ii) providing a second nucleic acid encoding a second polypeptide chain described herein; wherein the nucleic acids encoding the heavy and light chain variable regions are independently positioned on the first or second nucleic acid such they form an antigen binding domain for the antigen of interest;
and

c) expressing said nucleic acids encoding the first and second polypeptide chains in a host cell to produce a protein comprising said first and second polypeptide chains, respectively; and recovering a heterodimeric protein; and

d) evaluating the plurality of heterodimeric proteins produced for a biological activity of interest, e.g., an activity disclosed herein, for example ability to mediate ADCC.

In one aspect, the present disclosure provides a library of at least 5, 10, 20, 30, 50 hetero-multimeric proteins of the disclosure, wherein the proteins share domain arrangements but differ in the amino acid sequence of the variable domains of one, two or three of their antigen binding domains.

In one aspect, the present disclosure provides a library of at least 2, 3, 4, 5 or 10 hetero-multimeric proteins of the disclosure, wherein the proteins share the amino acid sequence of the variable domain of one, two or three of their antigen binding domains, but differ in domain arrangements.

In one aspect, provided is a pharmaceutical composition comprising a compound or composition described herein, and a pharmaceutically acceptable carrier.

In one aspect provided is the use of a polypeptide or composition of any one of the above claims as a medicament for the treatment of disease.

In one aspect provided is a method of treating a disease in a subject comprising administering to the subject a compound or composition described herein.

In one embodiment, the disease is a cancer or an infectious disease.

Any of the methods can further be characterized as comprising any step described in the application, including notably in the “Detailed Description of the Invention”). The invention further relates to a protein obtainable by any of present methods. The disclosure further relates to pharmaceutical or diagnostic formulations of the proteins of the present invention. The disclosure further relates to methods of using proteins in methods of treatment or diagnosis.

These and additional advantageous aspects and features of the invention may be further described elsewhere herein.

### BRIEF DESCRIPTION OF THE FIGURES

FIGS. 1A-1C show domain arrangements of anti-CD19 (A), anti-CD20 (B) and anti-EGFR (C) monovalent binding proteins in the F5 format having an ICb antigen binding domain whose antigen target is absent in cytotoxicity assays.

FIG. 2 shows the results of in vitro cytotoxicity assays which compared the ability of the CD19-F5 monovalent binding protein and a comparison full-length anti-CD19 antibody (human IgG1) containing the same variable regions to lyse Daudi tumor cells. The results show that the CD19-F5 monovalent binding protein is more potent in mediating cytotoxicity toward Daudi cells than full-length anti-CD19 antibody.

FIG. 3 shows the results of in vitro cytotoxicity assays which compared the ability of the GA101-F5 (anti-CD20) monovalent binding protein and a comparison full-length anti-CD20 antibody obinutuzumab (GA101; Gazyva™; Fc-optimized human IgG1) containing the same variable regions to lyse Daudi tumor cells. The results show that full-length anti-CD20 antibody obinutuzumab is more potent in mediating cytotoxicity toward Daudi cells than GA101-F5 monovalent binding protein in this setting. GA101-F6 monovalent binding protein whose Fc domain lacks FcγR binding did not mediate cytotoxicity.

FIG. 4 shows the results of survival of mice engrafted by i.v. with 5×10⁶ Raji tumor cells who received (one day after engraftment) one 25 μg/mouse i.v. administration of GA101-F5 (anti-CD20) monovalent binding protein, GA101-F6 (anti-CD20) monovalent binding protein lacking effector function, or full-length anti-CD20 antibody obinutuzumab (GA101; Gazyva™; Fc-optimized human IgG1). GA101-F5 monovalent binding protein displayed greater anti-tumor activity than full-length anti-CD20 antibody obinutuzumab, with 100% survival at 100 days post cell-injection. GA101-F6 monovalent binding protein whose Fc domain lacks FcγR binding did not significantly improve survival.

FIG. 5 shows the results of in vitro cytotoxicity assays which compared the ability of the EGFR-F5+ monovalent binding protein having amino acid substitutions in the Fc domain to enhance binding to human FcγRIIIa and a comparison full-length anti-EGFR antibody (cetuximab, human IgG1) containing the same variable regions to lyse A549 tumor cells. The results show that the EGFR-F5+ monovalent binding protein is more potent in mediating cytotoxicity toward tumor cells than full-length cetuximab. EGFR-F6 monovalent binding protein whose Fc domain lacks FcγR binding did not mediate cytotoxicity.

FIG. 6 shows domain arrangements of exemplary heterdimeric monovalent binding proteins that bind to human CD19, CD20, EGFR or PD-L1. The bonds shown (lines) between the two polypeptide chains are disulfide bonds.

### DETAILED DESCRIPTION OF THE INVENTION

### Definitions

As used in the specification, “a” or “an” may mean one or more. As used in the claim(s), when used in conjunction with the word “comprising”, the words “a” or “an” may mean one or more than one.

Where “comprising” is used, this can optionally be replaced by “consisting essentially of”, more optionally by “consisting of”.

As used herein, the term “antigen binding domain” or “ABD” refers to a domain comprising a three-dimensional structure capable of immunospecifically binding to an epitope. Thus, in one embodiment, said domain can comprise a hypervariable region, optionally a VH and/or VL domain of an antibody chain, optionally at least a VH domain. In another embodiment, the binding domain may comprise at least one complementarity determining region (CDR) of an antibody chain. In another embodiment, the binding domain may comprise a polypeptide domain from a non-immunoglobulin scaffold.

The term “antibody” herein is used in the broadest sense and specifically includes full-length monoclonal antibodies, polyclonal antibodies, monovalent antibodies (e.g., bispecific antibodies), and antibody fragments and derivatives, so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988). An “antibody fragment” comprises a portion of a full-length antibody, e.g. antigen-binding or variable regions thereof. Examples of antibody fragments include Fab, Fab′, F(ab)₂, F(ab)₂, F(ab)₃, Fv (typically the VL and VH domains of a single arm of an antibody), single-chain Fv (scFv), dsFv, Fd fragments (typically the VH and CH1 domain), and dAb (typically a VH domain) fragments; VH, VL, VhH, and V-NAR domains; minibodies, diabodies, triabodies, tetrabodies, and kappa bodies (see, e.g., III et al., Protein Eng 1997; 10: 949-57); camel IgG; IgNAR; and monovalent antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23, 1126-1136; WO2005040219, and published U.S. Patent Applications 20050238646 and 20020161201. The term “antibody derivative”, as used herein, comprises a full-length antibody or a fragment of an antibody, e.g. comprising at least antigen-binding or variable regions thereof, wherein one or more of the amino acids are chemically modified, e.g., by alkylation, PEGylation, acylation, ester formation or amide formation or the like. This includes, but is not limited to, PEGylated antibodies, cysteine-PEGylated antibodies, and variants thereof.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

By “constant region” as defined herein is meant an antibody-derived constant region that is encoded by one of the light or heavy chain immunoglobulin constant region genes. By “constant light chain” or “light chain constant region” as used herein is meant the region of an antibody encoded by the kappa (Cκ) or lambda (Cλ) light chains. The constant light chain typically comprises a single domain, and as defined herein refers to positions 108-214 of Cκ, or Cλ, wherein numbering is according to the EU index (Kabat et al., 1991, Sequences of Proteins of Immunological Interest, 5th Ed., United States Public Health Service, National Institutes of Health, Bethesda). By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cγ3 (CH3) and the hinge between Cγ1 and Cγ2. Although the boundaries of the Fc region may vary, the human IgG heavy chain Fc region is usually defined to comprise residues C226, P230 or A231 to its carboxyl-terminus, wherein the numbering is according to the EU index. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides include but are not limited to antibodies, Fc fusions and Fc fragments. Also, Fc regions according to the invention include variants containing at least one modification that alters (enhances or diminishes) an Fc associated effector function. Also, Fc regions according to the invention include chimeric Fc regions comprising different portions or domains of different Fc regions, e.g., derived from antibodies of different isotype or species.

By “variable region” as used herein is meant the region of an antibody that comprises one or more Ig domains substantially encoded by any of the VL (including Vκ and Vλ) and/or VH genes that make up the light chain (including κ and λ) and heavy chain immunoglobulin genetic loci respectively. A light or heavy chain variable region (VL and VH) consists of a “framework” or “FR” region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)), and as in Chothia. The framework regions of an antibody, that is the combined framework regions of the constituent light and heavy chains, serves to position and align the CDRs, which are primarily responsible for binding to an antigen.

The term “specifically binds to” means that an antibody or polypeptide can bind preferably in a competitive binding assay to the binding partner, as assessed using either recombinant forms of the proteins, epitopes therein, or native proteins present on the surface of isolated target cells. Competitive binding assays and other methods for determining specific binding are further described below and are well known in the art.

The term “affinity”, as used herein, means the strength of the binding of an antibody or polypeptide to an epitope. The affinity of an antibody is given by the dissociation constant K_{D}, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of surface plasmon resonance (SPR) screening (such as by analysis with a BIAcore™ SPR analytical device).

By “amino acid modification” herein is meant an amino acid substitution, insertion, and/or deletion in a polypeptide sequence. An example of amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y50W refers to a variant of a parent polypeptide, in which the tyrosine at position 50 is replaced with tryptophan. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of a polypeptide will exhibit 98%, 98%, or 99% homogeneity for polypeptides in the context of all present peptide species in the composition or at least with respect to substantially active peptide species in the context of proposed use.

In the context herein, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

The term “internalization”, used interchangeably with “intracellular internalization”, refers to the molecular, biochemical and cellular events associated with the process of translocating a molecule from the extracellular surface of a cell to the intracellular surface of a cell. The processes responsible for intracellular internalization of molecules are well-known and can involve, inter alia, the internalization of extracellular molecules (such as hormones, antibodies, and small organic molecules); membrane-associated molecules (such as cell-surface receptors); and complexes of membrane-associated molecules bound to extracellular molecules (for example, a ligand bound to a transmembrane receptor or an antibody bound to a membrane-associated molecule). Thus, “inducing and/or increasing internalization” refer to events wherein intracellular internalization is initiated and/or the rate and/or extent of intracellular internalization is increased.

The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils.

As used herein, the phrase “NK cells” refers to a sub-population of lymphocytes that is involved in non-conventional immunity. NK cells can be identified by virtue of certain characteristics and biological properties, such as the expression of specific surface antigens including CD56 and/or NKp46 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell K S and Colonna M). Humana Press. pp. 219-238 (2000).

### Producing Polypeptides

The antigen binding domains (ABDs) described herein can be readily derived from any of a variety of immunoglobulin scaffolds, notably from variable domains derived from antibodies (from immunoglobulin chains), for example in the form of associated V_{L} and V_{H} domains found on two polypeptide chains, or alternatively from suitable non-immunoglobulin scaffolds, for example that are functional when replacing V domains in the protein domain arrangements described herein. Typically, antibodies are initially obtained by immunization of a non-human animal, e.g., a mouse, rat, guinea pig or rabbit, with an immunogen comprising a polypeptide, or a fragment or derivative thereof, typically an immunogenic fragment, for which it is desired to obtain antibodies (e.g. a human polypeptide). The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988), the entire disclosure of which is herein incorporated by reference). Human antibodies may also be produced by using, for immunization, transgenic animals that have been engineered to express a human antibody repertoire (Jakobovitz et Nature 362 (1993) 255), or by selection of antibody repertoires using phage display methods. For example, a XenoMouse (Abgenix, Fremont, Calif.) can be used for immunization. A XenoMouse is a murine host that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference. Antibodies may also be produced by selection of combinatorial libraries of immunoglobulins, as disclosed for instance in (Ward et al. Nature, 341 (1989) p. 544, the entire disclosure of which is herein incorporated by reference). Phage display technology (McCafferty et al (1990) Nature 348:552-553) can be used to produce antibodies from immunoglobulin variable (V) domain gene repertoires from unimmunized donors. See, e.g., Griffith et al (1993) EMBO J. 12:725-734; U.S. Pat. Nos. 5,565,332; 5,573,905; 5,567,610; and 5,229,275). When combinatorial libraries comprise variable (V) domain gene repertoires of human origin, selection from combinatorial libraries will yield human antibodies.

Additionally, a wide range of antibodies are available in the scientific and patent literature, including DNA and/or amino acid sequences, or from commercial suppliers. Antibodies will typically be directed to a pre-determined antigen. Examples of antibodies include antibodies that recognize an antigen expressed by a target cell that is to be eliminated, for example a proliferating cell or a cell contributing to a disease pathology. Examples include antibodies that recognize tumor antigens, microbial (e.g. bacterial or parasite) antigens or viral antigens.

Variable domains and/or antigen binding domains can be selected based on the desired cellular target, and may include for example cancer antigens, bacterial or viral antigens, etc. As used herein, the term “bacterial antigen” includes, but is not limited to, intact, attenuated or killed bacteria, any structural or functional bacterial protein or carbohydrate, or any peptide portion of a bacterial protein of sufficient length (typically about 8 amino acids or longer) to be antigenic. Examples include gram-positive bacterial antigens and gram-negative bacterial antigens. In some embodiments the bacterial antigen is derived from a bacterium selected from the group consisting of Helicobacter species, in particular Helicobacter pyloris; Borrelia species, in particular Borrelia burgdorferi; Legionella species, in particular Legionella pneumophilia; Mycobacteria s species, in particular M. tuberculosis, M. avium, M. intracellulare, M. kansasii, M. gordonae; Staphylococcus species, in particular Staphylococcus aureus; Neisseria species, in particular N. gonorrhoeae, N. meningitidis; Listeria species, in particular Listeria monocytogenes; Streptococcus species, in particular S. pyogenes, S. agalactiae; S. faecalis; S. bovis, S. pneumonae; anaerobic Streptococcus species; pathogenic Campylobacter species; Enterococcus species; Haemophilus species, in particular Haemophilus influenzae; Bacillus species, in particular Bacillus anthracis; Corynebacterium species, in particular Corynebacterium diphtheriae; Erysipelothrix species, in particular Erysipelothrix rhusiopathiae; Clostridium species, in particular C. perfringens, C. tetani; Enterobacter species, in particular Enterobacter aerogenes, Klebsiella species, in particular Klebsiella 1S. pneumoniae, Pasteurella species, in particular Pasteurella multocida, Bacteroides species; Fusobacterium species, in particular Fusobacterium nucleatum; Streptobacillus species, in particular Streptobacillus moniliformis; Treponema species, in particular Treponema pertenue; Leptospira; pathogenic Escherichia species; and Actinomyces species, in particular Actinomyces israeli.

As used herein, the term “viral antigen” includes, but is not limited to, intact, attenuated or killed whole virus, any structural or functional viral protein, or any peptide portion of a viral protein of sufficient length (typically about 8 amino acids or longer) to be antigenic. Sources of a viral antigen include, but are not limited to viruses from the families: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., Ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviruses and rotaviruses); Bornaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), Hepatitis C; Norwalk and related viruses, and astroviruses). Alternatively, a viral antigen may be produced recombinantly.

As used herein, the terms “cancer antigen” and “tumor antigen” are used interchangeably and refer to antigens that are differentially expressed by cancer cells or are expressed by non-tumoral cells (e.g. immune cells) having a pro-tumoral effect (e.g. an immunosuppressive effect), and can thereby be exploited in order to target cancer cells. Cancer antigens are antigens which can potentially stimulate apparently tumor-specific immune responses. Some of these antigens are encoded, although not necessarily expressed, or expressed at lower levels or less frequently, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Still other cancer antigens can be expressed on immune cells capable of contributing to or mediating a pro-tumoral effect, e.g. cell that contributes to immune evasion, a monocyte or a macrophage, optionally a suppressor T cell, regulatory T cell, or myeloid-derived suppressor cell.

The cancer antigens are usually normal cell surface antigens which are either over-expressed or expressed at abnormal times, or are expressed by a targeted population of cells. Ideally the target antigen is expressed only on proliferative cells (e.g., tumor cells) or pro-tumoral cells (e.g. immune cells having an immunosuppressive effect), however this is rarely observed in practice. As a result, target antigens are in many cases selected on the basis of differential expression between proliferative/disease tissue and healthy tissue. Example of cancer antigens include: Receptor Tyrosine Kinase-like Orphan Receptor 1 (ROR1), Crypto, CD4, CD20, CD30, CD19, CD38, CD47, CD123, Glycoprotein NMB, CanAg, Her2 (ErbB2/Neu), a Siglec family member, for example CD22 (Siglec2) or CD33 (Siglec3), CD79, CD138, CD171, PSCA, L1-CAM, PSMA (prostate specific membrane antigen), BCMA, CD52, CD56, CD80, CD70, E-selectin, EphB2, Melanotransferrin, Mud 6 and TMEFF2. Examples of cancer antigens also include Immunoglobulin superfamily (IgSF) such as cytokine receptors, Killer-Ig Like Receptor, CD28 family proteins, for example, Killer-Ig Like Receptor 3DL2 (KIR3DL2), B7-H3, B7-H4, B7-H6, PD-L1, IL-6 receptor. Examples also include MAGE, MART-1/Melan-A, gp100, major histocompatibility complex class I-related chain A and B polypeptides (MICA and MICB), adenosine deaminase-binding protein (ADAbp), cyclophilin b, colorectal associated antigen (CRC)-C017-1A/GA733, protein tyrosine kinase 7(PTK7), receptor protein tyrosine kinase 3 (TYRO-3), nectins (e.g. nectin-4), major histocompatibility complex class I-related chain A and B polypeptides (MICA and MICB), proteins of the UL16-binding protein (ULBP) family, proteins of the retinoic acid early transcript-1 (RAET1) family, carcinoembryonic antigen (CEA) and its immunogenic epitopes CAP-1 and CAP-2, etv6, aml1, prostate specific antigen (PSA), T-cell receptor/CD3-zeta chain, MAGE-family of tumor antigens, GAGE-family of tumor antigens, anti-Müllerian hormone Type II receptor, delta-like ligand 4 (DLL4), DR5, ROR1 (also known as Receptor Tyrosine Kinase-Like Orphan Receptor 1 or NTRKR1 (EC 2.7.10.1), BAGE, RAGE, LAGE-1, NAG, GnT-V, MUM-1, CDK4, MUC family, VEGF, VEGF receptors, Angiopoietin-2, PDGF, TGF-alpha, EGF, EGF receptor, members of the human EGF-like receptor family, e.g., HER-2/neu, HER-3, HER-4 or a heterodimeric receptor comprised of at least one HER subunit, gastrin releasing peptide receptor antigen, Muc-1, CA125, integrin receptors, αvβ3 integrins, α5β1 integrins, αIIbβ3-integrins, PDGF beta receptor, SVE-cadherin, IL-8 receptor, hCG, IL-6 receptor, CSF1R (tumor-associated monocytes and macrophages), α-fetoprotein, E-cadherin, α-catenin, β-catenin and γ-catenin, p120ctn, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2, although this is not intended to be exhaustive. In one aspect, the antigen of interest is an antigen (e.g. any one of the antigens listed above) capable of undergoing intracellular internalization, for example when bound by a conventional human IgG1 antibody, either in the presence of absence of Fcγ receptor cells.

In one aspect, the antigen of interest or the tumor antigen is a CD19, CD20, EGFR or PD-L1 polypeptide; in one aspect, the monovalent binding protein comprises a VH and VL that form an antigen binding domain that binds CD19, CD20, EGFR or PD-L1, wherein the VH and VL comprising an amino acid sequence which is at least 60%, 70%, 80%, 85%, 90% or 95% identical to the sequence of respective anti-CD19, anti-CD20, anti-EGFR or anti-PD-L1 VH and VL described in the Examples herein, or that comprise the heavy and light chain CDR1, -2 and -3 of the anti-CD19, anti-CD20, anti-EGFR or anti-PD-L1 VH and VL described in the Examples herein.

In one aspect, provided is a monovalent binding protein that competes for binding to a human CD19, CD20, EGFR or PD-L1 polypeptide with a F5 or M5 protein disclosed in the Examples herein.

In the protein formats disclosed herein, antigen binding domains that bind human CD19, CD20, EGFR or PD-L1 can be conveniently produced by integrating CDRs and/or full VH and/or VL domains from the anti-CD19, CD20, EGFR or PD-L1 VH and VL domains disclosed herein.

In one embodiment, the antigen binding domain binds human PD-L1. The protein that comprises a PD-L1 binding domain as disclosed herein may be characterized by the ability to reduce the inhibitory activity of human PD-1 or neutralize the inhibitory activity of human PD-1.

“PD-1” refers to the protein Programmed Death 1 (PD-1) (also referred to as “Programmed Cell Death 1”), an inhibitory member of the CD28 family of receptors, that also includes CD28, CTLA-4, ICOS and BTLA. The complete human PD-1 sequence can be found under GenBank Accession No. U64863, shown as follows:

(SEQ ID NO: 58)
MQIPQAPWPVVWAVLQLGWRPGWFLDSPDRPWNPPTFFPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

PD-1 is expressed on activated B cells, T cells, and myeloid cells (Okazaki et al. (2002) Curr. Opin. Immunol. 14: 391779-82; Bennett et al. (2003) J Immunol 170:711-8), as well as in a limited portion of NK cells. The initial members of the family, CD28 and ICOS, were discovered by functional effects on augmenting T cell proliferation following the addition of monoclonal antibodies (Hutloff et al. (1999) Nature 397:263-266; Hansen et al. (1980) Immunogenics 10:247-260). Two ligands for PD-1 have been identified, PD-L1 and PD-L2, that have been shown to downregulate T cell activation upon binding to PD-1 (Freeman et al. (2000) J Exp Med 192:1027-34; Latchman et al. (2001) Nat Immunol 2:261-8; Carter et al. (2002) Eur J Immunol 32:634-43). Both PD-L1 and PD-L2 are B7 homologs that bind to PD-1, but do not bind to other CD28 family members.

The complete human PD-L1 sequence can be found under UniProtKB/Swiss-Prot, identifier Q9NZQ7-1, shown as follows:

(SEQ ID NO: 59)
MRIFAVFIFM TYWHLLNAFT VTVPKDLYVV EYGSNMTIEC
KFPVEKQLDL AALIVYWEME DKNIIQFVHG EEDLKVQHSS
YRQRARLLKD QLSLGNAALQ ITDVKLQDAG VYRCMISYGG
ADYKRITVKV NAPYNKINQR ILVVDPVTSE HELTCQAEGY
PKAEVIWTSS DHQVLSGKTT TTNSKREEKL FNVTSTLRIN
TTTNEIFYCT FRRLDPEENH TAELVIPELP LAHPPNERTH
LVILGAILLC LGVALTFIFR LRKGRMMDVK KCGIQDTNSK
KQSDTHLEET.

PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1. In the context of the present invention, “reduces the inhibitory activity of human PD-1”, “neutralizes PD-1”, “neutralizes PD-L1” or “neutralizes the inhibitory activity of human PD-1” refers to a process in which PD-1 is inhibited in its signal transduction capacity resulting from the interaction of PD-1 with PD-L1. An agent (e.g. protein) that neutralizes the inhibitory activity of PD-1 decreases, blocks, inhibits, abrogates or interferes with signal transduction resulting from the interaction of PD-1 with PD-L1. Such an agent can thereby reduce the negative co-stimulatory signal mediated by or through cell surface proteins expressed on T (or NK) lymphocytes, so as to enhance T-cell (or NK cell) effector functions such as proliferation, cytokine production and/or cytotoxicity.

Examples of anti-PD-L1 antibodies from which anti-PD-L1 binding domain can be obtained include VH and VL domains, or VH and VL CDRs thereof, of amino acid sequences of antibodies 3G10, 12A4, 10A5, 5F8, 10H10, 1612, 7H1, 11E6, 1267, and 13G4 shown in U.S. Pat. No. 7,943,743, the disclosure of which is incorporated herein by reference, or of any of the antibodies MPDL3280A (atezolizumab, Tecentriq™, see, e.g., U.S. Pat. No. 8,217,149, anti-PD-L1 from Roche/Genentech), MDX-1105 (anti-PD-L1 from Bristol-Myers Squibb), MSB0010718C (avelumab; anti-PD-L1 from Pfizer) and MEDI4736 (durvalumab; anti-PD-L1 from AstraZeneca). Exemplary VH and VL domain amino acid sequences that can be used in the antigen binding protein as anti-PD-L1 binding domains are also shown in the Table below.

TABLE
anti-PD-L1 VH and VL amino acid sequences

| | SEQ | |
|---|---|---|
| | ID | |
| Antibody | NO: | Amino acid sequence |
| MPDL3280A VH | 1 | EVQLVESGGGLVQPGGSLRLSCAASGFTFSDSWIHWVRQA |
| | | PGKGLEWVAWISPYGGSTYYADSVKGRFTISADTSKNTAYL |
| | | QMNSLRAEDTAVYYCARRHWPGGFDYWGQGTLVTVSS |
| MPDL3280A VL | 2 | DIQMTQSPSSLSASVGDRVTITCRASQDVSTAVAWYQQKPG |
| | | KAPKLLIYSASFLYSGVPSRFSGSGSGTDFTLTISSLQPEDFA |
| | | TYYCQQYLYHPATFGQGTKVEIK |
| MDX-1105 VH | 3 | QVQLVQSGAEVKKPGSSVKVSCKTSGDTFSTYAISWVRQAP |
| | | GQGLEWMGGIIPIFGKAHYAQKFQGRVTITADESTSTAYMEL |
| | | SSLRSEDTAVYFCARKFHFVSGSPFGMDVWGQGTTVTVSS |
| MDX-1105 VL | 4 | EIVLTQSPATLSLSPGERATLSCRASQSVSSYLAWYQQKPGQ |
| | | APRLLIYDASNRATGIPARFSGSGSGTDFTLTISSLEPEDFAV |
| | | YYCQQRSNWPTFGQGTKVEIK |
| MEDI4736 VH | 5 | EVQLVESGGGLVQPGGSLRLSCAASGFTFSRYWMSWVRQA |
| | | PGKGLEWVANIKQDGSEKYYVDSVKGRFTISRDNAKNSLYL |
| | | QMNSLRAEDTAVYYCAREGGWFGELAFDYWGQGTLVTVSS |
| MEDI4736 VL | 6 | EIVLTQSPGTLSLSPGERATLSCRASQRVSSSYLAWYQQKP |
| | | GQAPRLLIYDASSRATGIPDRFSGSGSGTDFTLTISRLEPEDF |
| | | AVYYCQQYGSLPWTFGQGTKVEIK |
| MSB0010718C | 7 | EVQLLESGGGLVQPGGSLRLSCAASGFTFSSYIMMWVRQAP |
| VH | | GKGLEWVSSIYPSGGITFYADTVKGRFTISRDNSKNTLYLQM |
| | | NSLRAEDTAVYYCARIKLGTVTTVDYWGQGTLVTVSS |
| MSB0010718C | 8 | QSALTQPASVSGSPGQSITISCTGTSSDVGGYNYVSWYQQH |
| VL | | PGKAPKLMIYDVSNRPSGVSNRFSGSKSGNTASLTISGLQAE |
| | | DEADYYCSSYTSSSTRVFGTGTKVTVL |

In a specific embodiment, an antigen binding protein can bind essentially the same epitope or determinant on a human PD-L1 protein as monoclonal antibody MPDL3280A, MDX-1105, MEDI4736 or MSB0010718; optionally the antigen binding protein comprises a hypervariable region of antibody MPDL3280A, MDX-1105, MEDI4736 or MSB0010718. In any of the embodiments herein, antibody MPDL3280A, MDX-1105, MEDI4736 or MSB0010718 can be characterized by its amino acid sequence and/or nucleic acid sequence encoding it. In one embodiment, the antigen binding protein comprises the heavy chain variable region of MPDL3280A, MDX-1105, MEDI4736 or MSB0010718s. According to one embodiment, an antigen binding protein comprises the three CDRs of the heavy chain variable region of MPDL3280A, MDX-1105, MEDI4736 or MSB0010718. Also provided is an antigen binding protein that further comprises the light chain variable region or one, two or three of the CDRs of the light chain variable region of the respective MPDL3280A, MDX-1105, MEDI4736 or MSB0010718 antibody. Optionally any one or more of said light or heavy chain CDRs may contain one, two, three, four or five or more amino acid modifications (e.g. substitutions, insertions or deletions).

In one aspect, the antigen binding protein is capable of inhibiting the interaction between PD-L1 and PD-1. In one aspect, in the presence of PD-L1-expressing target cells and PD-1-expressing cells (e.g. NK or T cells), the antigen binding protein is capable of neutralizing the inhibitory activity of PD-1 in the PD-1-expressing cells.

Optionally, the antigen binding protein, when incubated in soluble form with effector cells expressing PD-1 (e.g. PD-1⁺ T or NK cells), in the presence of PD-L1-expressing target cells, can induce the activation of the effector cells and/or lysis of PD-L1-expressing target cells, in particular via the activating signal(s) transmitted by at least in part by CD16, optionally wherein the antibody neutralizes PD-1 mediated inhibition of effector cell activation and/or target cell lysis, optionally wherein the antibody increases effector cell activation and/or target cell lysis to a degree comparable to that obtained by co-incubation with an antibody (e.g. a conventional antibody) that binds and inhibits PD-1, optionally wherein the antigen binding protein increases activation and/or lysis compared to an antigen binding protein that binds PD-L1 but does not inhibit the interaction between PD-L1 and PD-1 or a conventional full length antibody that binds PD-L1.

In one embodiment, the ABD (e.g. the VH-VL pair) that binds an antigen of interest is derived from (e.g. comprises the hypervariable region of, or comprises one, two, three, four, five or six of the CDRs of) a parental antibody that binds an antigen of interest (e.g. a murine antibody, a human antibody) which, when bound to its antigenic target (the antigen of interest on cells), increases or induces down-modulation or intracellular internalization of the antigen of interest. In one embodiment, the antigen of interest is a cancer antigen, e.g. one of the cancer antigens listed above known to internalize (e.g. Immunoglobulin superfamily (IgSF) members, for example cytokine receptor α or β chains, Killer-Ig Like Receptors, CD28 family proteins, B7-H3, B7-H4, B7-H6, KIR3DL2, PTK7, ROR1, L1-CAM, Siglec family members, EGF receptor and EGF-like receptor family members, EGFR, HER-2, integrins, anti-Müllerian hormone Type II receptor, CSF-1R, and others). In one embodiment, the antigen target is a polypeptide present on a cell present in the tumor environment and capable of mediating a pro-tumoral effect (e.g. a cancer-associated fibroblast (CAF), see e.g., Shiga et al. Cancers 2015, 7, 2443-2458). In one embodiment, the CAF is a cell that expresses α-SMA, tenascin-C, periostin, neuron glial antigen-2 (NG2), vimentin, desmin, platelet derived growth factor receptor-α and β (PDGFR α and β), and/or fibroblast specific protein-1 (FSP-1). In one embodiment, the antigen target is a polypeptide present on an immune cell capable of mediating a pro-tumoral effect, e.g. a monocyte or a macrophage, optionally a suppressor T cell, regulatory T cell, or myeloid-derived suppressor cell.

In exemplary embodiments, an ABD or pair of variable domains will bind an antigen expressed by a target cell that is to be eliminated (e.g., a tumor antigen, microbial (e.g. bacterial or parasitic) antigen, viral antigen, or antigen expressed on an immune cell that is contributing to inflammatory or autoimmune disease, and another ABD, variable domain or pair of complementary variable domains will bind to an antigen expressed on an immune cell, for example an immune effector cell, e.g. a cell surface receptor of an effector cells such as a T or NK cell. Examples of antigens expressed on immune cells, optionally immune effector cells, include antigens expressed on a member of the human lymphoid cell lineage, e.g. a human T cell, a human B cell or a human natural killer (NK) cell, a human monocyte, a human neutrophilic granulocyte or a human dendritic cell. Advantageously, such cells will have either a cytotoxic or an apoptotic effect on a target cell that is to be eliminated (e.g., that expresses a tumor antigen, microbial antigen, viral antigen, or antigen expressed on an immune cell that is contributing to inflammatory or autoimmune disease). Especially advantageously, the human lymphoid cell is a cytotoxic T cell or NK cell which, when activated, exerts a cytotoxic effect on the target cell. According to this embodiment, then, the cytotoxic activity of the human effector cells is recruited. According to another embodiment, the human effector cell is a member of the human myeloid lineage.

The ABDs or variable domains which are incorporated into the polypeptides can be tested for any desired activity prior to inclusion in a polypeptide. Once appropriate antigen binding domains having desired specificity and/or activity are identified, DNA encoding each variable domain can be placed, in suitable arrangements, in an appropriate expression vector(s), together with DNA encoding any elements such as an enzymatic recognition tag, or CH2 and CH3 domains and any other optional elements (e.g. DNA encoding a linker or hinge region) for transfection into an appropriate host(s). The host is then used for the recombinant production of the polypeptide chains that make up the monovalent protein.

An ABD or variable region pair derived from an antibody will generally comprise at minimum a hypervariable region sufficient to confer binding activity when present in the multimeric polypeptide. It will be appreciated that an ABD or variable region may comprise other amino acids or functional domains as may be desired, including but not limited to linker elements (e.g. linker peptides, constant domain derived sequences, hinges, or fragments thereof, each of which can be placed between a variable domain and a CH1, Cκ, CH2 or CH3 domain, or between other domains as needed).

In any embodiment, ABDs or variable regions can be obtained from a humanized antibody in which residues from a complementary-determining region (CDR) of a human antibody are replaced by residues from a CDR of the original antibody (the parent or donor antibody, e.g. a murine or rat antibody) while maintaining the desired specificity, affinity, and capacity of the original antibody. The CDRs of the parent antibody, some or all of which are encoded by nucleic acids originating in a non-human organism, are grafted in whole or in part into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536. An antigen binding domain can thus have non-human hypervariable regions or CDRs and human frameworks region sequences (optionally with back mutations).

Polypeptide chains will be arranged in one or more expression vectors so as to produce the polypeptides having the desired domains operably linked to one another. The host cell may be of mammalian origin or may be selected from COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof.

The polypeptides can then be produced in an appropriate host cell or by any suitable synthetic process and brought into contact under appropriate conditions for the heteromultimeric (e.g. dimer or trimer) polypeptide to form.

An isolated hetero-multimeric protein that binds an antigen of interest can be prepared according to different configurations, for example involving a first polypeptide chain and a second polypeptide chain. The first polypeptide chain can provide one variable domain that will, together with a complementary variable domain on a second polypeptide chain, form an antigen binding domain specific for the antigen of interest. One of the two polypeptide chains will comprise a CH1 domain and the other will comprise a Cκ domain, such that the two polypeptide chains will associate by CH1-Cκ heterodimerization, forming non-covalent interactions and optionally further interchain disulfide bonds between respective hinge domains and between complementary CH1 and CK domains, with a single multimeric protein being formed so long as CH/Cκ and VH/VK domains are chosen to give rise to a sole dimerization configuration.

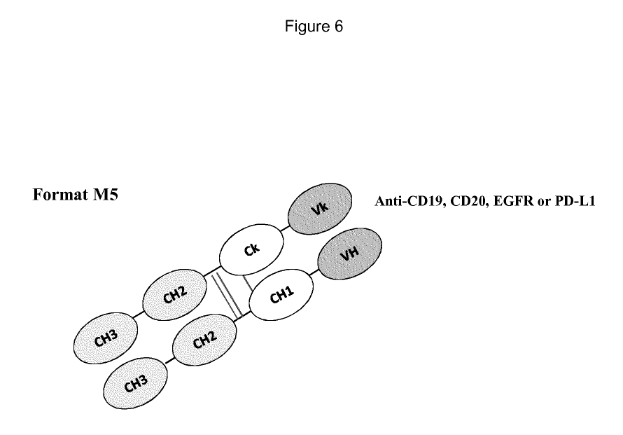

The resulting heterodimer can in another example have the configuration as follows (see also Examples of such proteins shown as format M5 shown in FIG. 6 and in Example 6):

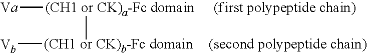

wherein one of Vₐ and V_{b} is a light chain variable domain and the other is a heavy chain variable domain, wherein Vₐ and V_{b} associate to form an antigen binding domain that binds an antigen of interest. In one embodiment, a hinge region is present on each polypeptide chain between the (CH1 or CK) unit and the Fc domain. In one embodiment, the hinge regions of the first and second polypeptide associate by disulfide bonding.

The heterodimeric protein can be characterized by binding Fcγ receptor(s) or more generally immune effector cells solely via its Fc domain, and not via an ABD comprising one or more antibody variable domains. That is, the heterodimeric protein binds immune effector cells (when such are not target cells to be depleted) via its Fc domain thereby mediating ADCC. The heterodimeric protein is highly potent despite lacking additional antigen binding domains (e.g. Ig variable domains) that bind immune cell surface proteins, and which, for example, serve to bring immune effector cells into contact with target cells and/or to further enhance effector cell cytotoxicity. The heterodimeric protein may optionally comprise any additional amino acid sequences (e.g., protein domains) fused to the C-terminus of (or C-terminal of) the Fc domains of a polypeptide chain; such additional sequences are however such domains other than antibody variable domains or optionally other than protein domains that bind a cell-surface protein. For example, an additional sequence can be a polypeptide sequence that binds a non-cellular biological target. The non-cellular target can be, e.g., a soluble protein such as a cytokine. In one example, the additional sequence is a portion of a receptor protein that is capable of binding a soluble protein such as an immunosuppressive cytokine.

A VH or VL can be linked to a CH1 or CK constant domain via a linker peptide. Examples of linkers include, for example, linkers derived from antibody hinge regions, an amino sequence RTVA.

Generally, any domain can optionally be fused to another domain on a polypeptide chain via linking amino acid sequences. Peptide linkers may comprise a length of at least 4 residues, at least 5 residues, at least 10 residues, at least 15 residues, at least 20 residues, at least 25 residues, at least 30 residues or more. In other embodiments, the linkers comprise a length of between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-22 residues, between 2-24 residues, between 2-26 residues, between 2-28 residues, between 2-30 residues, between 2 and 50 residues, or between 10 and 50 residues.

In one embodiment, each CH1 and CK domain (a full CH1 or CK domain or a fragment thereof) is linked or fused to an Fc domain (e.g. to a CH2 domain) via a hinge region or fragment thereof.

In one embodiment, each CH1 and CK domain is linked or fused to an Fc domain via a hinge region (or fragment thereof) derived form a hinge domain of a human IgG1 antibody. For example a hinge domain may comprise the amino acid sequence (one letter code): THTCSSCPAPELL (SEQ ID NO: 9), or an amino acid sequence at least 60%, 70%, 80% or 90% identical thereto, optionally wherein one or both cysteines are deleted or substituted by a different amino acid residue.

In one embodiment, the hinge region (or fragment thereof) is derived from a Cμ2-C Cμ3 hinge domain of a human IgM antibody. For example a hinge domain may comprise the amino acid sequence (one letter code): NASSMCVPSPAPELL (SEQ ID NO: 10), or an amino acid sequence at least 60%, 70%, 80% or 90% identical thereto, optionally wherein one or both cysteines are deleted or substituted by a different amino acid residue.

Polypeptide chains that dimerize and associate with one another via non-covalent bonds may or may not additionally be bound by an interchain disulfide bond formed between respective CH1 and Cκ domains, and/or between respective hinge domains on the chains. CH1, Cκ and/or hinge domains (or other suitable linking amino acid sequences) can optionally be configured such that interchain disulfide bonds are formed between chains such that the desired pairing of chains is favored and undesired or incorrect disulfide bond formation is avoided. For example, when two polypeptide chains to be paired each possess a CH1 or Cκ adjacent to a hinge domain, the polypeptide chains can be configured such that the number of available cysteines for interchain disulfide bond formation between respective CH1/Cκ-hinge segments is reduced (or is entirely eliminated). For example, the amino acid sequences of respective CH1, Cκ and/or hinge domains can be modified to remove cysteine residues in both the CH1/Cκ and the hinge domain of a polypeptide; thereby the CH1 and Cκ domains of the two chains that dimerize will associate via non-covalent interaction(s).

In another example, the CH1 or Cκ domain adjacent to (e.g., N-terminal to) a hinge domain comprises a cysteine capable of interchain disulfide bond formation, and the hinge domain which is placed at the C-terminus of the CH1 or Cκ comprises a deletion or substitution of one or both cysteines of the hinge (e.g. Cys 239 and Cys 242, as numbered for human IgG1 hinge according to Kabat). In one embodiment, the hinge region (or fragment thereof) comprise the amino acid sequence (one letter code): THTSPPSPAPELL (SEQ ID NO: 11), or an amino acid sequence at least 60%, 70%, 80% or 90% identical thereto.

In another example, the CH1 or Cκ domain adjacent to (e.g., N-terminal to) a hinge domain comprises a deletion or substitution at a cysteine residue capable of interchain disulfide bond formation, and the hinge domain placed at the C-terminus of the CH1 or Cκ comprises one or both cysteines of the hinge (e.g. Cys 239 and Cys 242, as numbered for human IgG1 hinge according to Kabat). In one embodiment, the hinge region (or fragment thereof) comprises the amino acid sequence (one letter code): THTCSSCPAPELL (SEQ ID NO: 9), or an amino acid sequence at least 60%, 70%, 80% or 90% identical thereto.

In another example, a hinge region is derived from an IgM antibody. In such embodiments, the CH1/Cκ pairing mimics the Cμ2 domain homodimerization in IgM antibodies. For example, the CH1 or Cκ domain adjacent (e.g., N-terminal to) a hinge domain comprises a deletion or substitution at a cysteine capable of interchain disulfide bond formation, and an IgM hinge domain which is placed at the C-terminus of the CH1 or Cκ comprises one or both cysteines of the hinge. In one embodiment, the hinge region (or fragment thereof) comprises the amino acid sequence (one letter code): THTCSSCPAPELL (SEQ ID NO: 9), or an amino acid sequence at least 60%, 70%, 80% or 90% identical thereto.

Constant region domains can be derived from any suitable human antibody, including, the constant heavy (CH1) and light (Cκ) domains, hinge domains, CH2 and CH3 domains. “CH1” generally refers to positions 118-220 according to the EU index as in Kabat. “CH2” generally refers to positions 237-340 according to the EU index as in Kabat, and “CH3” generally refers to positions 341-447 according to the EU index as in Kabat.

A “hinge” or “hinge region” or “antibody hinge region” herein refers to the flexible polypeptide or linker between the first and second constant domains of an antibody. Structurally, the IgG CH1 domain ends at EU position 220, and the IgG CH2 domain begins at residue EU position 237. Thus for an IgG the hinge generally includes positions 221 (D221 in IgG1) to 236 (G236 in IgG1), wherein the numbering is according to the EU index as in Kabat. References to specific amino acid residues within constant region domains found within the polypeptides shall be, unless otherwise indicated or as otherwise dictated by context, be defined according to Kabat, in the context of an IgG antibody.

CH2 and CH3 domains which may be present in the subject antibodies or monovalent proteins can be derived from any suitable antibody. Such CH2 and CH3 domains can be used as wild-type domains or may serve as the basis for a modified CH2 or CH3 domain. Optionally the CH2 and/or CH3 domain is of human origin or may comprise that of another species (e.g., rodent, rabbit, non-human primate) or may comprise a modified or chimeric CH2 and/or CH3 domain, e.g., one comprising portions or residues from different CH2 or CH3 domains, e.g., from different antibody isotypes or species antibodies.

CH2 and/or CH3 domain (or Fc domain comprising same) may be wild-type domains or may comprise one or more amino acid modifications (e.g. amino acid substitutions) which increase binding to human CD16 and optionally another receptor such as another Fcγ receptor and/or FcRn. Optionally, the modifications will not substantially decrease or abolish the ability of the Fc-derived polypeptide to bind to neonatal Fc receptor (FcRn), e.g. human FcRn. Typical modifications include modified human IgG1-derived constant regions comprising at least one amino acid modification (e.g. substitution, deletions, insertions), and/or altered types of glycosylation, e.g., hypofucosylation. Such modifications can affect interaction with Fcγ receptors: FcγRI (CD64), FcγRII (CD32), and FcγRIII (CD16). FcγRI (CD64), FcγRIIA (CD32A) and FcγRIII (CD16) are activating (i.e., immune system enhancing) receptors while FcγRIIB (CD32B) is an inhibiting (i.e., immune system dampening) receptor. A modification may, for example, increase binding of the Fc domain to FcγRIIIa on effector (e.g. NK) cells and/or decrease binding to FcγRIIB. Examples of modifications are provided in PCT publication no. WO2014/044686, the disclosure of which is incorporated herein by reference. Specific mutations in IgG1 which affect (enhance) FcγRIIIa or FcRn binding are also set forth below.

| | | | Effector | Effect of |
|---|---|---|---|---|
| Isotype | Species | Modifications | Function | Modification |
| IgG1 | Human | T250Q/M428L | Increased | Increased |
| | | | binding to FcRn | half-life |
| IgG1 | Human | 1M252Y/S254T/ | Increased | Increased |
| | | T256E + H433K/ | binding to FcRn | half-life |
| | | N434F | | |
| IgG1 | Human | E333A | Increased | Increased |
| | | | binding to | ADCC and |
| | | | FcγRIIIa | CDC |
| IgG1 | Human | S239D/I332E; | Increased | Increased |
| | | S239D/A330L/ | binding to | ADCC |
| | | I332E | FcγRIIIa | |
| IgG1 | Human | S239D/I332E/ | Increased | Increased |
| | | G236A | FcγRIIa/FcγRIIb | macrophage |
| | | | ratio | phagocytosis |

In some embodiments, the monovalent protein comprises a variant Fc region comprising e at least one amino acid modification (for example, possessing 1, 2, 3, 4, 5, 6, 7, 8, 9, or more amino acid modifications) in the CH2 and/or CH3 domain of the Fc region, wherein the modification enhances binding to a human CD16 polypeptide. In other embodiments, the monovalent protein comprises at least one amino acid modification (for example, 1, 2, 3, 4, 5, 6, 7, 8, 9, or more amino acid modifications) in the CH2 domain of the Fc region from amino acids 237-341, or within the lower hinge-CH2 region that comprises residues 231-341. In some embodiments, the monovalent protein comprises at least two amino acid modifications (for example, 2, 3, 4, 5, 6, 7, 8, 9, or more amino acid modifications), wherein at least one of such modifications is within the CH3 region and at least one such modifications is within the CH2 region. Encompassed also are amino acid modifications in the hinge region. In one embodiment, encompassed are amino acid modifications in the CH1 domain, optionally in the upper hinge region that comprises residues 216-230 (Kabat EU numbering). Any suitable functional combination of Fc modifications can be made, for example any combination of the different Fc modifications which are disclosed in any of U.S. Pat. Nos. 7,632,497; 7,521,542; 7,425,619; 7,416,727; 7,371,826; 7,355,008; 7,335,742; 7,332,581; 7,183,387; 7,122,637; 6,821,505 and 6,737,056; and/or in PCT Publications Nos. WO2011/109400; WO 2008/105886; WO 2008/002933; WO 2007/021841; WO 2007/106707; WO 06/088494; WO 05/115452; WO 05/110474; WO 04/1032269; WO 00/42072; WO 06/088494; WO 07/024249; WO 05/047327; WO 04/099249 and WO 04/063351; and/or in Lazar et al. (2006) Proc. Nat. Acad. Sci. USA 103(11): 405-410; Presta, L. G. et al. (2002) Biochem. Soc. Trans. 30(4):487-490; Shields, R. L. et al. (2002) J. Biol. Chem. 26; 277(30):26733-26740 and Shields, R. L. et al. (2001) J. Biol. Chem. 276(9):6591-6604).

In some embodiments, the monovalent protein comprises an Fc domain comprising at least one amino acid modification (for example, 1, 2, 3, 4, 5, 6, 7, 8, 9, or more amino acid modifications) relative to a wild-type Fc region, such that the molecule has an enhanced binding affinity for human CD16 relative to the same molecule comprising a wild-type Fc region, optionally wherein the variant Fc region comprises a substitution at any one or more of positions 221, 239, 243, 247, 255, 256, 258, 267, 268, 269, 270, 272, 276, 278, 280, 283, 285, 286, 289, 290, 292, 293, 294, 295, 296, 298, 300, 301, 303, 305, 307, 308, 309, 310, 311, 312, 316, 320, 322, 326, 329, 330, 332, 331, 332, 333, 334, 335, 337, 338, 339, 340, 359, 360, 370, 373, 376, 378, 392, 396, 399, 402, 404, 416, 419, 421, 430, 434, 435, 437, 438 and/or 439 (Kabat EU numbering).

In one embodiment, the monovalent protein comprises an Fc domain comprising at least one amino acid modification (for example, 1, 2, 3, 4, 5, 6, 7, 8, 9, or more amino acid modifications) relative to a wild-type Fc region, such that the molecule has enhanced binding affinity for human CD16 relative to a molecule comprising a wild-type Fc region, optionally wherein the variant Fc region comprises a substitution at any one or more of positions 239, 298, 330, 332, 333 and/or 334 (e.g. S239D, S298A, A330L, I332E, E333A and/or K334A substitutions), optionally wherein the variant Fc region comprises a substitution at residues S239 and I332, e.g. a S239D and I332E substitution (Kabat EU numbering).

In some embodiments, the monovalent antigen binding protein comprises an Fc domain comprising altered glycosylation patterns that increase binding affinity for human CD16. Such carbohydrate modifications can be accomplished by, for example, by expressing a nucleic acid encoding the monovalent protein in a host cell with altered glycosylation machinery. Cells with altered glycosylation machinery are known in the art and can be used as host cells in which to express recombinant antibodies to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 06/133148; WO 03/035835; WO 99/54342, each of which is incorporated herein by reference in its entirety. In one aspect, the monovalent protein contains one or more hypofucosylated constant regions. Such monovalent protein may comprise an amino acid alteration or may not comprise an amino acid alteration and/or may be expressed or synthesized or treated under conditions that result in hypofucosylation. In one aspect, a monovalent protein composition comprises a monovalent protein described herein, wherein at least 20, 30, 40, 50, 60, 75, 85, 90, 95% or substantially all of the antibody species in the composition have a constant region comprising a core carbohydrate structure (e.g. complex, hybrid and high mannose structures) which lacks fucose. In one embodiment, provided is a monovalent protein composition which is free of antibodies comprising a core carbohydrate structure having fucose. The core carbohydrate will preferably be a sugar chain at Asn297.

Optionally, a monovalent antigen binding protein comprising a dimeric Fc domain can be characterized by having a binding affinity to a human CD16 polypeptide that is within 1 log-, 2-log or 3-log of that of a conventional human IgG1 antibody, e.g., as assessed by surface plasmon resonance.

Optionally a monovalent antigen binding protein comprising a dimeric Fc domain can be characterized by a Kd for binding (monovalent) to a human CD16 polypeptide of less than 10⁻⁵ M (10 μmolar), optionally less than 10⁻⁶ M (1 μmolar), as assessed by surface plasmon resonance (e.g. SPR measurements performed on a Biacore T100 apparatus (Biacore GE Healthcare), with monovalent antigen binding protein immobilized on a Sensor Chip CM5 and serial dilutions of soluble CD16 polypeptide injected over the immobilized bispecific antibodies.

In some embodiments, the Fc domains can comprise modifications to enhance CH3-CH3 association through use of a “knob-into-holes” approach. In this approach, the CH3 domain interface of the dimeric Fc region is mutated so that the first and second polypeptide chains preferentially form heterodimers. These mutations create altered charge polarity across the Fc dimer interface such that co-expression of electrostatically matched Fc chains support favorable attractive interactions thereby promoting desired Fc heterodimer formation, whereas unfavorable repulsive charge interactions suppress unwanted Fc homodimer formation. For example one chain comprises a T366W substitution and the other chain comprises a T366S, L368A and Y407V substitution, see, e.g. Ridgway et al (1996) Protein Eng., 9, pp. 617-621; Atwell (1997) J. Mol. Biol., 270, pp. 26-35; and WO2009/089004, the disclosures of which are incorporated herein by reference. In another approach, one chain comprises a F405L substitution and the other chain comprises a K409R substitution, see, e.g., Labrijn et al. (2013) Proc. Natl. Acad. Sci. U.S.A., 110, pp. 5145-5150. In another approach, one chain comprises T350V, L351Y, F405A, and Y407V substitutions and the other chain comprises T350V, T366S, K392L, and T394W substitutions, see, e.g. Von Kreudenstein et al., (2013) mAbs 5:646-654. In another approach, one chain comprises both K409D and K392D substitutions and the other chain comprises both D399K and E356K substitutions, see, e.g. Gunasekaran et al., (2010) J. Biol. Chem. 285:19637-19646. In another approach, one chain comprises D221E, P228E and L368E substitutions and the other chain comprises D221R, P228R, and K409R substitutions, see, e.g. Strop et al., (2012) J. Mol. Biol. 420: 204-219. In another approach, one chain comprises S364H and F405A substitutions and the other chain comprises Y349T and, T394F substitutions, see, e.g. Moore et al., (2011) mAbs 3: 546-557. In another approach, one polypeptide chain comprises a H435R substitution and the other chain optionally may or may not comprise a substitution, see, e.g. U.S. Pat. No. 8,586,713.

In any embodiment herein, the Fc domain may be characterized as comprising mammalian antibody-type N-linked glycosylation at residue N297 (Kabat EU numbering).

In one embodiment, a heterodimer protein (e.g. comprising a wild type Fc domain) comprises a first polypeptide chain comprising the amino acid sequence shown in SEQ ID NO: 12, below, or an amino acid sequence at least 60%, 70%, 80%, 90%, 95% or 99% identical thereto. The first polypeptide chain can further be specified to comprise a heavy or light chain variable region fused to the start (N-terminus) thereof.

(SEQ ID NO: 12)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGECDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVD
KSRWQQGNVFSCSVMHEALHNHYTQKSLSLSPGK

In one embodiment, a heterodimer protein (e.g. comprising a wild type Fc domain) comprises a second polypeptide chain comprising the amino acid sequence shown in SEQ ID NO: 13, below, or an amino acid sequence at least 60%, 70%, 80%, 90%, 95% or 99% identical thereto. The second polypeptide chain can further be specified to comprise a heavy or light chain variable region fused to the start (N-terminus) thereof. When the variable region fused to the N-terminus of the first chain is a VH domain, the variable region fused to the N-terminus of the second chain is a VL domain. When the variable region fused to the N-terminus of the first chain is a VL domain, the variable region fused to the N-terminus of the second chain is a VH domain.

(SEQ ID NO: 13)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYSSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In one embodiment, a heterodimer protein comprising an Fc domain modified to enhance CD16A binding comprises a first polypeptide chain comprising the amino acid sequence shown in SEQ ID NO: 14, below, or an amino acid sequence at least 60%, 70%, 80%, 90%, 95% or 99% identical thereto. The first polypeptide chain can further be specified to comprise a heavy or light chain variable region fused to the start (N-terminus) thereof.

(SEQ ID NO: 14)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGECDKTHTCPPCPAPELLGGPDVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPEEKTISKAKGQPREPQVYTLPPSREEMTKNQV
SLTCLVKGFYPSDIAVEWESNGQPENNYKTTPPVLDSDGSFFLYSKLTVD
KSRWQQGNVFSCSVMHEALHNHYTQKSLSLSPGK

In one embodiment, a heterodimer protein comprising an Fc domain modified to enhance CD16A binding comprises a second polypeptide chain comprising the amino acid sequence shown in SEQ ID NO: 15, below, or an amino acid sequence at least 60%, 70%, 80%, 90%, 95% or 99% identical thereto. The second polypeptide chain can further be specified to comprise a heavy or light chain variable region fused to the start (N-terminus) thereof. When the variable region fused to the N-terminus of the first chain is a VH domain, the variable region fused to the N-terminus of the second chain is a VL domain. When the variable region at the N-terminus of the first chain is a VL domain, the variable region at the N-terminus of the second chain is a VH domain.

(SEQ ID NO: 15)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPDVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPEEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

The amino acid sequences of the first and second polypeptide chains of exemplary heterodimers are shown below for proteins that bind CD19, CD20, EGFR and PD-L1, respectively.

### Anti-CD19 Heterodimer Protein (Wild Type Fc Domain):

Polypeptide chain 1:
(SEQ ID NO: 16)
DIQLTQSPASLAVSLGQRATISCKASQSVDYDGDSYLNWYQQIPGQPPKL
LIYDASNLVSGIPRFSGSGSGTDFTLNIHPVEKVDAATYHCQQSTEDPWT
FGGGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGECDKTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK
Polypeptide chain 2:
(SEQ ID NO: 17)
QVQLQQSGAELVRPGSSVKISCKASGYAFSSYWMNWVKQRPGQGLEWIGQ
IWPGDGDTNYNGKFKGKATLTADESSSTAYMQLSSLASEDSAVYFCARRE
TTTVGRYYYAMDYWGQGTTVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPRE
EQYSSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPGK

### Anti-CD20 Heterodimer Protein (Wild Type Fc Domain):

Polypeptide chain 1:
(SEQ ID NO: 18)
DIVMTQTPLSLPVTPGEPASISCRSSKSLLHSNGITYLYWYLQKPGQSPQ
LLIYQMSNLVSGVPDRFSGSGSGTDFTLKISRVEAEDVGVYYCAQNLELP
YTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGECDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
Polypeptide chain 2:
(SEQ ID NO: 19)
QVQLVQSGAEVKKPGSSVKVSCKASGYAFSYSWINWVRQAPGQGLEWMGR
IFPGDGDTDYNGKFKGRVTITADKSTSTAYMELSSLRSEDTAVYYCARNV
FDGYWLVYWGQGTLVTVSSASTKGPSVFPLAPSSKSTSGGTAALGCLVKD
YFPEPVTVSWNSGALTSGVHTFPAVLQSSGLYSLSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYSST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK

### Anti-EGFR Heterodimer Protein (Wild Type Fc Domain):

Polypeptide chain 1:
(SEQ ID NO: 20)
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGECDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLY
SKLTVDKSRWQQGNVFSCSVMHEALHNHYTQKSLSLSPGK
Polypeptide chain 2:
(SEQ ID NO : 21)
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYSS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK

### Anti-PD-L1 Heterodimer Protein (Wild Type Fc Domain):

Polypeptide chain 1:
(SEQ ID NO : 22)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGECDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLY
SKLTVDKSRWQQGNVFSCSVMHEALHNHYTQKSLSLSPGK
Polypeptide chain 2:
(SEQ ID NO: 23)
QVQLVQSGAEVKKPGSSVKVSCKTSGDTFSTYAISWVRQAPGQGLEWMGG
IIPIFGKAHYAQKFQGRVTITADESTSTAYMELSSLRSEDTAVYFCARKF
HFVSGSPFGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYSSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK

### Anti-CD19 Heterodimer Protein (Fc Domain Modified to Enhance CD16A Binding):

Polypeptide chain 1:
(SEQ ID NO: 24)
DIQLTQSPASLAVSLGQRATISCKASQSVDYDGDSYLNWYQQIPGQPPKLLIYDASNLVSGIPPRFSG
SGSGTDFTLNIHPVEKVDAATYHCQQSTEDPWTFGGGTKLEIKRTVAAPSVFIFPPSDEQLKSGTASV
VCLLNNFYPREAKVQWKVDNALQSGNSQESVTEQDSKDSTYSLSSTLTLSKADYEKHKVYACEVTHQG
LSSPVTKSFNRGECDKTHTCPPCPAPELLGGPDVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPEEKTISKAKGQ
PREPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
Polypeptide chain 2:
(SEQ ID NO: 25)
QVQLQQSGAELVRPGSSVKISCKASGYAFSSYWMNWVKQRPGQGLEWIGQIWPGDGDTNYNGKFKGKA
TLTADESSSTAYMQLSSLASEDSAVYFCARRETTTVGRYYYAMDYWGQGTTVTVSSASTKGPSVFPLA
PSSKSTSGGTAALGCLVKDYFPEPVTVSWNSGALTSGVHTFPAVLQSSGLYSLSSVVTVPSSSLGTQT
YICNVNHKPSNTKVDKRVEPKSCDKTHTCPPCPAPELLGGPDVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPEE
KTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

### Anti-CD20 Heterodimer Protein (Fc Domain Modified to Enhance CD16A Binding):

Polypeptide chain 1:
(SEQ ID NO: 26)
DIVMTQTPLSLPVTPGEPASISCRSSKSLLHSNGITYLYWYLQKPGQSPQLLIYQMSNLVSGVPDRFS
GSGSGTDFTLKISRVEAEDVGVYYCAQNLELPYTFGGGTKVEIKRTVAAPSVFIFPPSDEQLKSGTAS
VVCLLNNFYPREAKVQWKVDNALQSGNSQESVTEQDSKDSTYSLSSTLTLSKADYEKHKVYACEVTHQ
GLSSPVTKSFNRGECDKTHTCPPCPAPELLGGPDVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPEEKTISKAKG
QPREPQVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGK
Polypeptide chain 2:
(SEQ ID NO: 27)
QVQLVQSGAEVKKPGSSVKVSCKASGYAFSYSWINWVRQAPGQGLEWMGRIFPGDGDTDYNGKFKGRV
TITADKSTSTAYMELSSLRSEDTAVYYCARNVFDGYWLVYWGQGTLVTVSSASTKGPSVFPLAPSSKS
TSGGTAALGCLVKDYFPEPVTVSWNSGALTSGVHTFPAVLQSSGLYSLSSVVTVPSSSLGTQTYICNV
NHKPSNTKVDKRVEPKSCDKTHTCPPCPAPELLGGPDVFLFPPKPKDTLMISRTPEVTCVVVDVSHED
PEVKFNWYVDGVEVHNAKTKPREEQYNSTYRVVSVLTVLHQDWLNGKEYKCKVSNKALPAPEEKTISK
AKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK

### Anti-EGFR Heterodimer Protein (Fc Domain Modified to Enhance CD16A Binding):

Polypeptide chain 1:
(SEQ ID NO: 28)
DILLTQSPVILSVSPGERVSFSCRASQSIGTNIHWYQQRTNGSPRLLIKYASESISGIPSRFSGSGSG
TDFTLSINSVESEDIADYYCQQNNNWPTTFGAGTKLELRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPV
TKSFNRGECDKTHTCPPCPAPELLGGPDVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPEEKTISKAKGQPREPQ
VYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
Polypeptide chain 2:
(SEQ ID NO: 29)
QVQLKQSGPGLVQPSQSLSITCTVSGFSLTNYGVHWVRQSPGKGLEWLGVIWSGGNTDYNTPFTSRLS
INKDNSKSQVFFKMNSLQSNDTAIYYCARALTYYDYEFAYWGQGTLVTVSAASTKGPSVFPLAPSSKS
TSGGTAALGCLVKDYFPEPVTVSWNSGALTSGVHTFPAVLQSSGLYSLSSVVTVPSSSLGTQTYICNV
NHKPSNTKVDKRVEPKSCDKTHTCPPCPAPELLGGPDVFLFPPKPKDTLMISRTPEVTCVVVDVSHED
PEVKFNWYVDGVEVHNAKTKPREEQYNSTYRVVSVLTVLHQDWLNGKEYKCKVSNKALPAPEEKTISK
AKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK

### Anti-PD-L1 Heterodimer Protein (Fc Domain Modified to Enhance CD16A Binding):

Polypeptide chain 1:
(SEQ ID NO: 30)
EIVLTQSPATLSLSPGERATLSCRASQSVSSYLAWYQQKPGQAPRLLIYDASNRATGIPARFSGSGSG
TDFTLTISSLEPEDFAVYYCQQRSNWPTFGQGTKVEIKRTVAAPSVFIFPPSDEQLKSGTASVVCLLN
NFYPREAKVQWKVDNALQSGNSQESVTEQDSKDSTYSLSSTLTLSKADYEKHKVYACEVTHQGLSSPV
TKSFNRGECDKTHTCPPCPAPELLGGPDVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPEEKTISKAKGQPREPQ
VYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
Polypeptide chain 2:
(SEQ ID NO: 31)
QVQLVQSGAEVKKPGSSVKVSCKTSGDTFSTYAISWVRQAPGQGLEWMGGIIPIFGKAHYAQKFQGRV
TITADESTSTAYMELSSLRSEDTAVYFCARKFHFVSGSPFGMDVWGQGTTVTVSSASTKGPSVFPLAP
SSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPELLGGPDVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPEEK
TISKAKGQPREPQVYTLPPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK

In one embodiment, a multispecific heterodimeric antigen binding protein comprises a first polypeptide chain comprising the amino acid sequence of the Polypeptide chain 1 of the Anti-CD19 heterodimer protein shown above, optionally wherein the light chain variable domain is substituted by a different variable domain (e.g. a different light chain variable domain; a variable domain from a parental antibody that binds an antigen of interest), and a second polypeptide chain comprising the amino acid sequence of the Polypeptide chain 2 of the Anti-CD19 heterodimer protein shown above, optionally wherein the heavy chain variable domain is substituted by a different variable domain (e.g. a different heavy chain variable domain; a variable domain from a parental antibody that binds an antigen of interest).

In one embodiment, a multispecific heterodimeric CD19-binding protein comprises a first polypeptide chain comprising the amino acid sequence of the Polypeptide chain 1 of the Anti-CD19 heterodimer protein shown above, and a second polypeptide chain comprising the amino acid sequence of the Polypeptide chain 2 of the Anti-CD19 heterodimer protein shown above. In one embodiment, a multispecific heterodimeric CD19-binding protein comprises a first polypeptide chain comprising an amino acid sequence at least 60%, 70%, 80%, 90%, 95% or 98% identical to the amino acid sequence of the Polypeptide chain 1 of the Anti-CD19 heterodimer protein shown above, and a second polypeptide chain comprising an amino acid sequence at least 60%, 70%, 80%, 90%, 95% or 98% identical to the amino acid sequence of the Polypeptide chain 2 of the Anti-CD19 heterodimer protein shown above.

In one embodiment, a multispecific heterodimeric CD20-binding protein comprises a first polypeptide chain comprising the amino acid sequence of the Polypeptide chain 1 of the Anti-CD20 heterodimer protein shown above, and a second polypeptide chain comprising the amino acid sequence of the Polypeptide chain 2 of the Anti-CD20 heterodimer protein shown above. In one embodiment, a multispecific heterodimeric CD20-binding protein comprises a first polypeptide chain comprising an amino acid sequence at least 60%, 70%, 80%, 90%, 95% or 98% identical to the amino acid sequence of the Polypeptide chain 1 of the Anti-CD20 heterodimer protein shown above, and a second polypeptide chain comprising an amino acid sequence at least 60%, 70%, 80%, 90%, 95% or 98% identical to the amino acid sequence of the Polypeptide chain 2 of the Anti-CD20 heterodimer protein shown above.

In one embodiment, a multispecific heterodimeric EGFR-binding protein comprises a first polypeptide chain comprising the amino acid sequence of the Polypeptide chain 1 of the Anti-EGFR heterodimer protein shown above, and a second polypeptide chain comprising the amino acid sequence of the Polypeptide chain 2 of the Anti-EGFR heterodimer protein shown above. In one embodiment, a multispecific heterodimeric EGFR-binding protein comprises a first polypeptide chain comprising an amino acid sequence at least 60%, 70%, 80%, 90%, 95% or 98% identical to the amino acid sequence of the Polypeptide chain 1 of the Anti-EGFR heterodimer protein shown above, and a second polypeptide chain comprising an amino acid sequence at least 60%, 70%, 80%, 90%, 95% or 98% identical to the amino acid sequence of the Polypeptide chain 2 of the Anti-EGFR heterodimer protein shown above.

In one embodiment, a multispecific heterodimeric PD-L1-binding protein comprises a first polypeptide chain comprising the amino acid sequence of the Polypeptide chain 1 of the Anti-PD-L1 heterodimer protein shown above, and a second polypeptide chain comprising the amino acid sequence of the Polypeptide chain 2 of the Anti-PD-L1 heterodimer protein shown above. In one embodiment, a multispecific heterodimeric PD-L1-binding protein comprises a first polypeptide chain comprising an amino acid sequence at least 60%, 70%, 80%, 90%, 95% or 98% identical to the amino acid sequence of the Polypeptide chain 1 of the Anti-PD-L1 heterodimer protein shown above, and a second polypeptide chain comprising an amino acid sequence at least 60%, 70%, 80%, 90%, 95% or 98% identical to the amino acid sequence of the Polypeptide chain 2 of the Anti-PD-L1 heterodimer protein shown above.

In one embodiment, a multispecific heterodimeric CD19, CD20, EGFR or PD-L1-binding protein comprises a first polypeptide chain comprising an amino acid sequence at least 60%, 70%, 80%, 90%, 95% or 98% identical to the amino acid sequence of the Polypeptide chain 1 of the respective Anti-CD19, -CD20, -EGFR or -PD-L1 heterodimer protein shown above, wherein the percentage sequence identity is computed excluding the variable domain, and a second polypeptide chain comprising an amino acid sequence at least 60%, 70%, 80%, 90%, 95% or 98% identical to the amino acid sequence of the Polypeptide chain 2 of the respective Anti-CD19, -CD20, -EGFR or -PD-L1 heterodimer protein shown above, wherein the percentage sequence identity is computed excluding the variable domain. In one embodiment, a multispecific heterodimeric CD19, CD20, EGFR or PD-L1-binding protein comprises (a) a first polypeptide chain (or “Frag1”) of the respective anti-CD19, -CD20, -EGFR or -PD-L1 having the amino acid sequence shown in Example 6 (a polypeptide chain of SEQ ID NOS: 42-57), optionally without the N-terminal leader amino acid sequence, or an amino acid sequence at least 60%, 70%, 80%, 90%, 95% or 98% identical thereto, and (b) a second polypeptide chain (or “Frag2”) of the respective anti-CD19, -CD20, -EGFR or -PD-L1 having the amino acid sequence shown in Example 6 (a polypeptide chain of SEQ ID NOS: 42-57), optionally without the N-terminal leader amino acid sequence, or an amino acid sequence at least 60%, 70%, 80%, 90%, 95% or 98% identical thereto.

In one embodiment, the disclosure provides methods of making a heterodimeric protein (e.g. any heterodimeric protein described herein), comprising:

a) providing a first nucleic acid encoding a first polypeptide chain described herein (e.g., a polypeptide chain comprising a heavy chain variable domain (VH) fused to a CH1 of CK constant region and an Fc domain or portion thereof);

b) providing a second nucleic acid encoding a second polypeptide chain described herein (e.g., a polypeptide chain comprising a light chain variable domain (VL) fused at its C-terminus to a CH1 or CK constant region selected to be complementary to the CH1 or CK constant region of the first polypeptide chain such that the first and second polypeptides form a CH1-CK heterodimer in which the VH of the first polypeptide chain and the VL of the second polypeptide form an antigen binding domain, and an Fc domain or portion thereof); and

c) expressing said first and second nucleic acids in a host cell to produce a protein comprising said first and second polypeptide chains, respectively; and recovering a heterodimeric protein comprising a dimeric Fc domain capable of binding human CD16. Optionally, the heterodimeric protein produced represents at least 20%, 25% or 30% of the total proteins (e.g. bispecific proteins) prior to purification. Optionally step (c) comprises loading the protein produced onto an affinity purification support, optionally an affinity exchange column, optionally a Protein-A support or column, and collecting the heterodimeric protein; and/or loading the protein produced (or the protein collected following loading onto an affinity exchange or Protein A column) onto an ion exchange column; and collecting the heterodimeric fraction.

By virtue of their ability to be produced in standard cell lines and standardized methods with high yields, the proteins of the disclosure also provide a convenient tool for screening for the most effective variable regions to incorporate into a monovalent antigen binding protein. In one aspect, the present disclosure provides a method for identifying or evaluating candidate variable regions for use in a heterodimeric protein, comprising the steps of:

a) providing a plurality of nucleic acid pairs, wherein each pair includes one nucleic acid encoding a candidate heavy chain variable region and one nucleic acid encoding a candidate light chain variable region, for each of a plurality of heavy and light chain variable region pairs (e.g., obtained from different antibodies binding the same or different antigen(s) of interest);

b) for each of the plurality nucleic acid pairs, making a heterodimeric protein by:

(i) producing a first nucleic acid encoding a first polypeptide chain comprising a candidate heavy chain variable domain (VH) fused to a CH1 or CK constant region, and an Fc domain or portion thereof;

(ii) producing a second nucleic acid encoding a second polypeptide chain comprising a candidate light chain variable domain (VL) fused to a CH1 or CK constant region, selected to be complementary to the CH1 or CK constant region of the first polypeptide chain such that the first and second polypeptides form a CH1-CK heterodimer in which the candidate heavy and light chain variable domains form an antigen binding domain, and an Fc domain or portion thereof; and

(iii) expressing said nucleic acids encoding the first and second polypeptide chains in a host cell to produce a protein comprising said first and second polypeptide chains, respectively; and recovering a heterodimeric protein comprising a dimeric Fc domain capable of binding human CD16; and

c) evaluating the plurality of heterodimeric proteins produced for a biological activity of interest, e.g., an activity disclosed herein. Optionally, the heterodimeric protein produced represents at least 20%, 25% or 30% of the total proteins prior to purification. Optionally the recovering step in (iii) comprises loading the protein produced onto an affinity purification support, optionally an affinity exchange column, optionally a Protein-A support or column, and collecting the heterodimeric protein; and/or loading the protein produced (or the protein collected following loading onto a affinity exchange or Protein A column) onto an ion exchange column; and collecting the heterodimeric fraction.

In the methods for identifying or evaluating candidate variable regions, it will be appreciated that the candidate variable regions can be for example from an antigen that binds an antigen of interest.

In one aspect of the any of the embodiments herein, recovering a heterodimeric or heterotrimer protein can comprise introducing the protein to a solid phase so as to immobilize the protein. The immobilized protein can then subsequently be eluted. Generally, the solid support may be any suitable insoluble, functionalized material to which the proteins can be reversibly attached, either directly or indirectly, allowing them to be separated from unwanted materials, for example, excess reagents, contaminants, and solvents. Examples of solid supports include, for example, functionalized polymeric materials, e.g., agarose, or its bead form Sepharose®, dextran, polystyrene and polypropylene, or mixtures thereof; compact discs comprising microfluidic channel structures; protein array chips; pipet tips; membranes, e.g., nitrocellulose or PVDF membranes; and microparticles, e.g., paramagnetic or non-paramagnetic beads. In some embodiments, an affinity medium will be bound to the solid support and the protein will be indirectly attached to solid support via the affinity medium. In one aspect, the solid support comprises a protein A affinity medium or protein G affinity medium. A “protein A affinity medium” and a “protein G affinity medium” each refer to a solid phase onto which is bound a natural or synthetic protein comprising an Fc-binding domain of protein A or protein G, respectively, or a mutated variant or fragment of an Fc-binding domain of protein A or protein G, respectively, which variant or fragment retains the affinity for an Fc-portion of an antibody. Protein A and Protein G are bacterial cell wall proteins that have binding sites for the Fc portion of mammalian IgG. The capacity of these proteins for IgG varies with the species. In general, IgGs have a higher affinity for Protein G than for Protein A, and Protein G can bind IgG from a wider variety of species. The affinity of various IgG subclasses, especially from mouse and human, for Protein A varies more than for Protein G. Protein A can, therefore, be used to prepare isotypically pure IgG from some species. When covalently attached to a solid matrix, such as cross-linked agarose, these proteins can be used to capture and purify antigen-protein complexes from biochemical solutions. Commercially available products include, e.g., Protein G, A or L bonded to agarose or sepharose beads, for example EZview™ Red Protein G Affinity Gel is Protein G covalently bonded to 4% Agarose beads (Sigma Aldrich Co); or POROS® A, G, and CaptureSelect® HPLC columns (Invitrogen Inc.). Affinity capture reagents are also described, for example, in the Antibody Purification Handbook, Biosciences, publication No. 18-1037-46, Edition AC, the disclosure of which is hereby incorporated by reference).

Once the monovalent antigen binding protein is produced it can be assessed for biological activity. In one aspect of any embodiment herein, where a protein binds an antigen on a target cell to be eliminated, a monovalent protein is capable of inducing mediating ADCC, e.g., causing the activation of an immune effector cell (e.g. an NK cell, a T cell), when the protein is incubated in the presence of the effector cell and a target cell that expresses the antigen of interest). In one aspect of any embodiment herein, a monovalent antigen binding protein is capable of mediating activation of an immune effector cell when the protein is incubated in the presence of the effector cell and a target cell that expresses the antigen of interest). Optionally, effector cell activation is characterized by increased expression of a cell surface marker of activation, e.g. CD107, CD69, etc. Activity can be measured for example by bringing target cells and effector cells into contact with one another, in presence of the monovalent antigen binding polypeptide. In one example, aggregation of target cells and effector cells is measured. In another example, the monovalent antigen binding protein may, for example, be assessed for the ability to cause a measurable increase in any property or activity known in the art as associated with NK cell activity, respectively, such as marker of cytotoxicity (CD107) or cytokine production (for example IFN-γ or TNF-α), increases in intracellular free calcium levels, the ability to lyse target cells in a redirected killing assay, etc. In one embodiment of any of the methods of identifying, evaluating or making a protein, the method comprises a step of evaluating the monovalent antigen binding protein for its ability to induce or increase the activity of immune cells (e.g. by assessing a marker of activation or cytotoxicity, cytokine production, ability to lyse a target cell, etc.), when incubated in the presence of the immune cells and target cells expressing an antigen of interest bound by an ABD of the monovalent antigen binding protein (e.g. a cancer antigen). In one embodiment, the immune cells express CD16. In one embodiment, the cells are NK cells.

In the presence of target cells (target cells expressing the antigen of interest) and effector cells that express the activating receptor bound by the protein, the monovalent antigen binding protein will be capable of causing an increase in a property or activity associated with effector (e.g. NK cell, T cell) cell activity (e.g. activation of NK cell cytotoxicity, CD107 expression, IFNγ production) in vitro. For example, a monovalent antigen binding protein of the disclosure can be selected for the ability to increase an NK or T cell activity by more than about 20%, preferably with at least about 30%, at least about 40%, at least about 50%, or more compared to that achieved with the same effector: target cell ratio with the same NK or T cells and target cells that are not brought into contact with the monovalent antigen binding protein, as measured by an assay of NK or T cell activity, e.g., a marker of activation of NK cell cytotoxicity, CD107 or CD69 expression, IFNγ production, a classical in vitro chromium release test of cytotoxicity. Examples of protocols for activation and cytotoxicity assays are described in the Examples herein, as well as for example, in Pessino et al, J. Exp. Med, 1998, 188 (5): 953-960; Sivori et al, Eur J Immunol, 1999. 29:1656-1666; Brando et al, (2005) J. Leukoc. Biol. 78:359-371; El-Sherbiny et al, (2007) Cancer Research 67(18):8444-9; and Nolte-'t Hoen et al, (2007) Blood 109:670-673).

In one aspect of the any of the embodiments herein, evaluating proteins for a characteristic of interest comprises evaluating the proteins for one or more properties selected from the group consisting of: binding to an antigen of interest, binding to an antigen on an immune effector cell, binding to a tumor, viral or bacterial antigen, binding to an FcRn receptor, binding to human CD16 and/or other Fc-domain mediated effector function(s), agonistic or antagonistic activity at an antigen of interest to which the proteins binds, ability to modulate the activity (e.g. cause the death of) a cell expressing the antigen of interest, ability to direct a lymphocyte to a cell expressing the antigen of interest, intracellular internalization when bound to a target cell, ability to activate a lymphocyte in the presence and/or absence of a cell expressing the antigen of interest, NK cell activation, stability or half-life in vitro or in vivo, production yield, purity within a composition, and susceptibility to aggregate in solution.

In one aspect, the present disclosure provides a method for identifying or evaluating a protein, comprising the steps of:

(a) providing nucleic acid(s) encoding a protein described herein;

(b) expressing said nucleic acid(s) in a host cell to produce said protein, respectively; and recovering said protein; and

(c) evaluating the protein produced for a biological activity of interest, e.g., an activity disclosed herein, the ability to mediate the lysis of target cells (that express antigen of interest). In one embodiment, a plurality of different monovalent antigen binding proteins are produced and evaluated.

In one embodiment, the step (c) comprises:

(i) testing the ability of the protein to cause effector cells (e.g. NK cells, T cells) to mediate the lysis of target cells, when incubated with such effector cells in the presence of target cells (that express antigen of interest). Optionally, step (i) is followed by a step comprising: selecting a protein (e.g., for further development, for use as a medicament) that mediates the lysis of target cells.

### Uses of Compounds

In one aspect, provided is the use of any of the compounds defined herein, particularly the inventive monovalent antigen binding proteins or antibodies and/or cells which express same for the manufacture of a pharmaceutical preparation for the treatment, prevention or diagnosis of a disease in a mammal in need thereof. Provided also are the use any of the compounds defined above as a medicament or an active component or active substance in a medicament. In a further aspect the invention provides methods for preparing a pharmaceutical composition containing a compound as defined herein, to provide a solid or a liquid formulation for administration orally, topically, or by injection. Such a method or process at least comprises the step of mixing the compound with a pharmaceutically acceptable carrier.

In one aspect, provided is a method to treat, prevent or more generally affect a predefined condition in an individual, notably a human individual, or to detect a certain condition by using or administering a monovalent protein described herein, or a (pharmaceutical) composition comprising same.

The polypeptides described herein can be used to prevent or treat disorders that can be treated with antibodies, such as cancers, solid tumors, hematological malignancies, infections such as viral or microbial/bacterial infections, and inflammatory or autoimmune disorders.

In one embodiment, the polypeptides described herein can be used to prevent or treat a solid tumor, optionally a cancer selected from the group consisting of: carcinoma, including that of the bladder, head and neck, breast, colon, kidney, liver, lung, ovary, prostate, pancreas, stomach, cervix, thyroid and skin, including squamous cell carcinoma.

In one embodiment, herein the an antigen of interest to which a protein of the disclosure binds is expressed on the surface of a target cell present in a solid tumor or tumor environment, e.g. a cell present in the tumor tissue or in tumor-adjacent tissue that is to be eliminated.

In one embodiment, the an antigen of interest to which a protein of the disclosure binds is expressed on the surface of a malignant cell of a type cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sézary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukemia (T-Lbly/T-ALL).

In one embodiment, the inventive monovalent polypeptides described herein can be used to prevent or treat a cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma. Other exemplary disorders that can be treated according to the invention include hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sézary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukaemia (T-Lbly/T-ALL).

In one aspect, the methods of treatment comprise administering to an individual a monovalent antigen binding protein of the disclosure in a therapeutically effective amount. A therapeutically effective amount may be any amount that has a therapeutic effect in a patient having a disease or disorder (or promotes, enhances, and/or induces such an effect in at least a substantial proportion of patients with the disease or disorder and substantially similar characteristics as the patient).

The monovalent antigen binding proteins of the disclosure can be included in kits. The kits may optionally further contain any number of polypeptides and/or other compounds, e.g., 1, 2, 3, 4, or any other number of monovalent proteins and/or other compounds. It will be appreciated that this description of the contents of the kits is not limiting in any way. For example, the kit may contain other types of therapeutic compounds. Optionally, the kits also include instructions for using the polypeptides, e.g., detailing the herein-described methods.

The invention also provides pharmaceutical compositions comprising the subject monovalent antigen binding proteins and optionally other compounds as defined above. A monovalent protein and optionally another compound may be administered in purified form together with a pharmaceutical carrier as a pharmaceutical composition. The form depends on the intended mode of administration and therapeutic or diagnostic application. The pharmaceutical carrier can be any compatible, nontoxic substance suitable to deliver the compounds to the patient. Pharmaceutically acceptable carriers are well known in the art and include, for example, aqueous solutions such as (sterile) water or physiologically buffered saline or other solvents or vehicles such as glycols, glycerol, oils such as olive oil or injectable organic esters, alcohol, fats, waxes, and inert solids A pharmaceutically acceptable carrier may further contain physiologically acceptable compounds that act for example to stabilize or to increase the absorption of the compounds Such physiologically acceptable compounds include, for example, carbohydrates, such as glucose, sucrose or dextrans, antioxidants, such as ascorbic acid or glutathione, chelating agents, low molecular weight proteins or other stabilizers or excipients One skilled in the art would know that the choice of a pharmaceutically acceptable carrier, including a physiologically acceptable compound, depends, for example, on the route of administration of the composition Pharmaceutically acceptable adjuvants, buffering agents, dispersing agents, and the like, may also be incorporated into the pharmaceutical compositions. Non-limiting examples of such adjuvants include by way of example inorganic and organic adjuvants such as alum, aluminum phosphate and aluminum hydroxide, squalene, liposomes, lipopolysaccharides, double stranded (ds) RNAs, single stranded (s-s) DNAs, and TLR agonists such as unmethylated CpG's.

Monovalent antigen binding proteins according to the invention can be administered parenterally. Preparations of the compounds for parenteral administration must be sterile. Sterilization is readily accomplished by filtration through sterile filtration membranes, optionally prior to or following lyophilization and reconstitution. The parenteral route for administration of compounds is in accord with known methods, e.g. injection or infusion by intravenous, intraperitoneal, intramuscular, intraarterial, or intralesional routes. The compounds may be administered continuously by infusion or by bolus injection. A typical composition for intravenous infusion could be made up to contain 100 to 500 ml of sterile 0.9% NaCl or 5% glucose optionally supplemented with a 20% albumin solution and 1 mg to 10 g of the compound, depending on the particular type of compound and its required dosing regimen. Methods for preparing parenterally administrable compositions are well known in the art.

### EXAMPLES

### Example 1

### Monovalent Antigen Binding Formats with Dimeric Fc Domains

New protein constructions with dimeric Fc domains (wild type human CH2 and CH3 domains) were developed that bind FcRn and either had low/lack of binding to FcγR (including CD16) or that retained binding to FcγRs (including CD16) with a binding affinity similar to that of conventional human IgG1 antibodies.

Initial protein formats were based on functional bispecific antigen binding proteins having the domain arrangement of the F5 or F6 format shown in PCT application number PCT/EP2016/064537, filed 23 Jun. 2016 (Innate Pharma). However, in this example the proteins differed in that one antigen binding domain was selected to bind an antigen known to be absent on cells used in cytotoxicity assays (immune cells and tumor target cells), while the other antigen binding domain was selected to bind an antigen (CD19) present on a target cell to be depleted (a tumor cell). The different polypeptide formats were tested and compared to investigate the functionality of heteromultimeric proteins comprising polypeptide chains with complementary (V_{H}-(CH1/Cκ)-CH2-CH3) or (Vκ-(CH1 or Cκ)-CH2-CH3) units. One of both of the CH3 domains was fused at its C-terminus to a variable domain(s) (a single variable domain that associates with a variable domain on a third polypeptide chain. The different chains associated by CH1-Cκ dimerization to form a disulfide linked heterotrimer with a dimeric Fc domain.

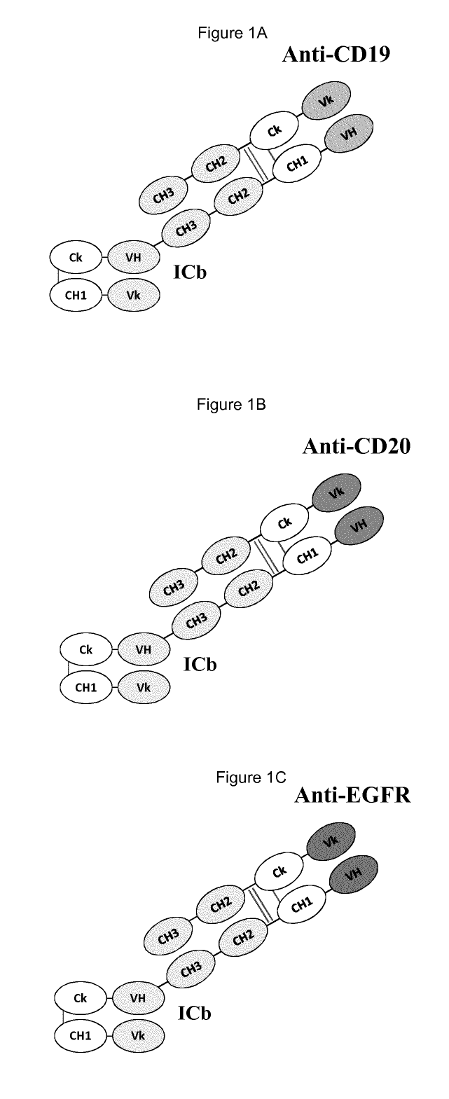

Different constructs were made using the variable domains DNA and amino acid sequences derived from the anti-CD19 antibody VH and VL domains shown below, and from the antibody bezlotoxumab specific for the Clostridum difficile toxin B (trade name Zinplava™). Domains structures of the resulting protein shown in FIG. 1A. “ICb” is used to refer to the bezlotoxumab-derived antigen binding domain (which is non-functional in this experimental setting due to absence of its target antigen).

Coding sequences were generated by direct synthesis and/or by PCR. PCR was performed using the PrimeSTAR MAX DNA polymerase (Takara) and PCR products were purified from 1% agarose gel using the NucleoSpin gel and PCR clean-up kit (Macherey-Nagel). Once purified the PCR products were quantified prior to the In-Fusion ligation reaction which was performed as described in the manufacturer's protocol (ClonTech). The plasmids were obtained after a miniprep preparation run on an EVO200 (Tecan) using the Nucleospin 96 plasmid kit (Macherey-Nagel). Plasmids were then sequenced for sequence confirmation before to transfecting the CHO cell line.

CHO cells were grown in the CD-CHO medium (Invitrogen) complemented with phenol red and 6 mM GlutaMax. The day before the transfection, cells were counted and seeded at 175,000 cells/ml. For the transfection, cells (200,000 cells/transfection) were prepared as described in the AMAXA SF cell line kit (AMAXA) and nucleofected using the DS137 protocol with the Nucleofector 4D device. All the transfections were performed using 300 ng of verified plasmids. After transfection, cells were seeded into 24 well plates in pre-warmed culture medium. After 24 hours, hygromycin B was added in the culture medium (200 μg/ml). Protein expression was monitored after one week in culture. Cells expressing the proteins were then sub-cloned to obtain the best producers. Sub-cloning was performed using 96 flat-bottom well plates in which the cells are seeded at one cell per well into 200 μl of culture medium complemented with 200 μg/ml of hygromycin B. Cells were left for three weeks before testing the clone's productivity.

Recombinant proteins which contain an IgG1-Fc fragment were purified using Protein-A beads (-rProteinA Sepharose fast flow, GE Healthcare). Briefly, cell culture supernatants were concentrated, clarified by centrifugation and injected onto Protein-A columns to capture the recombinant Fc containing proteins. Proteins were eluted at acidic pH (citric acid 0.1M pH 3), and the eluate immediately neutralized using TRIS-HCL pH 8.5 and dialyzed against 1×PBS. Other recombinant proteins were purified by size exclusion chromatography (SEC).

Anti-CD19-VK
(SEQ ID NO: 32)
GACATTCAGCTGACCCAATCTCCAGCTTCTTTGGCTGTGTCTCTAGGGCAGAGGGCCACCATCTCCTG
CAAGGCCAGCCAAAGTGTTGATTATGATGGTGATAGTTATTTGAACTGGTACCAACAGATACCAGGAC
AGCCACCCAAACTCCTCATCTATGATGCATCCAATCTAGTATCTGGGATTCCACCCAGGTTTAGTGGC
AGTGGGTCTGGGACAGACTTCACCCTCAACATCCATCCTGTGGAGAAGGTGGATGCTGCAACCTATCA
CTGTCAGCAAAGTACTGAGGACCCTTGGACGTTCGGTGGAGGCACCAAGCTGGAAATCAAA
Anti-CD19-VK
(SEQ ID NO: 33)
DIQLTQSPASLAVSLGQRATISCKASQSVDYDGDSYLNWYQQIPGQPPKLLIYDASNLVSGIPPRFSG
SGSGTDFTLNIHPVEKVDAATYHCQQSTEDPWTFGGGTKLEIK
Anti-CD19-VH
(SEQ ID NO: 34)
CAGGTTCAGCTGCAGCAGTCTGGGGCTGAGCTGGTGCGGCCTGGGTCCTCAGTGAAGATTTCCTGCAA
AGCATCTGGCTACGCATTCAGTAGCTACTGGATGAACTGGGTGAAGCAGAGGCCTGGACAGGGTCTTG
AGTGGATTGGACAGATTTGGCCTGGAGATGGTGATACTAACTACAACGGAAAGTTCAAGGGCAAGGCC
ACACTGACTGCAGACGAATCCTCCAGCACAGCCTACATGCAGCTCAGCAGCCTGGCCTCTGAGGACTC
TGCGGTCTATTTCTGTGCAAGACGAGAAACGACCACTGTCGGGCGTTATTACTATGCTATGGACTACT
GGGGTCAAGGAACCACAGTCACCGTCTCCTCA
Anti-CD19-VH
(SEQ ID NO: 35)
QVQLQQSGAELVRPGSSVKISCKASGYAFSSYWMNWVKQRPGQGLEWIGQIWPGDGDTNYNGKFKGKA
TLTADESSSTAYMQLSSLASEDSAVYFCARRETTTVGRYYYAMDYWGQGTTVTVSS

Format 5 (F5): CD19-F5-ICb

The domain structure of the trimeric F5 polypeptide is shown in FIG. 1A, wherein the interchain bonds between hinge domains (indicated in the figures between CH1/Cκ and CH2 domains on a chain) and interchain bonds between the CH1 and Cκ domains are interchain disulfide bonds. The VH-VL pair that binds Clostridum difficile toxin B is referred to as “ICb”. The heterotrimer is made up of:

(1) a first (central) polypeptide chain having domains arranged as follows (N- to C-termini):

V_{H}^{anti-C19}-CH1-CH2-CH3-V_{H}^{ICb}-Cκ

and

(2) a second polypeptide chain having domains arranged as follows (N- to C-termini): Vκ^{anti-CD19}-CK-CH2-CH3

and

(3) a third polypeptide chain having domains arranged as follows (N- to C-termini):

Vκ^{ICb}-CH1.

Proteins were cloned, produced and purified. Proteins was purified from cell culture supernatant by affinity chromatography using prot-A beads and analyzed and purified by SEC. The amino acid sequences of the three polypeptide chains, including the leader sequences, are shown below, as well as their domain arrangement indicated below each amino acid sequence. The sequences shown include leader sequences (underlined); it will be appreciated that amino sequences can also be represented as mature sequences without the indicated leader sequences.

CD19-F5_Frag1_VK_chain:
(SEQ ID NO: 36)
MSVPTQVLGLLLLWLTDARCDIQLTQSPASLAVSLGQRATISCKASQSVDYDGDSYLNWYQQIPGQPP
KLLIYDASNLVSGIPPRFSGSGSGTDFTLNIHPVEKVDAATYHCQQSTEDPWTFGGGTKLEIKRTVAA
PSVFIFPPSDEQLKSGTASVVCLLNNFYPREAKVQWKVDNALQSGNSQESVTEQDSKDSTYSLSSTLT
LSKADYEKHKVYACEVTHQGLSSPVTKSFNRGECDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK
Domain arrangement: Leader-VK-CK-Hinge-CH2-CH3
CD19-F5-ICb_Frag2_VH_chain:
(SEQ ID NO: 37)
MEWSWVFLFFLSVTTGVHSQVQLQQSGAELVRPGSSVKISCKASGYAFSSYWMNWVKQRPGQGLEWIG
QIWPGDGDTNYNGKFKGKATLTADESSSTAYMQLSSLASEDSAVYFCARRETTTVGRYYYAMDYWGQG
TTVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPGSTGS
EVQLVQSGAEVKKSGESLKISCKGSGYSFTSYWIGWVRQMPGKGLEWMGIFYPGDSSTRYSPSFQGQV
TISADKSVNTAYLQWSSLKASDTAMYYCARRRNWGNAFDIWGQGTMVTVSSRTVAAPSVFIFPPSDEQ
LKSGTASVVCLLNNFYPREAKVQWKVDNALQSGNSQESVTEQDSKDSTYSLSSTLTLSKADYEKHKVY
ACEVTHQGLSSPVTKSFNRGEC
Domain arrangement: Leader-VH-CH1-Hinge-CH2-CH3-STGS-Icb-VH-CK
CD19-F5/F6-ICb_Frag3_Light_chain:
(SEQ ID NO: 38)
MSVPTQVLGLLLLWLTDARCEIVLTQSPGTLSLSPGERATLSCRASQSVSSSYLAWYQQKPGQAPRLL
IYGASSRATGIPDRFSGSGSGTDFTLTISRLEPEDFAVYYCQQYGSSTWTFGQGTKVEIKASTKGPSV
FPLAPSSKSTSGGTAALGCLVKDYFPEPVTVSWNSGALTSGVHTFPAVLQSSGLYSLSSVVTVPSSSL
GTQTYICNVNHKPSNTKVDKRVEPKSCDKTHS
Domain arrangement: Leader-Icb-VK-CH1

Format 6 (F6): CD19-F6-ICb

The domain structure of heterotrimeric F6 polypeptide is shown in FIG. 1A. The F6 protein is the same as F5 in the preceding example, but F6 differs in that F6 contains a N297S substitution (not indicated in FIG. 1A) to avoid N-linked glycosylation to remove capacity to bind FcγR and to mediate effector functions. Proteins were cloned, produced and purified. Proteins were purified from cell culture supernatant by affinity chromatography using prot-A beads and analyzed and purified by SEC. The amino acid sequences of the three chains of the F6 protein are shown below.

CD19-F6_Frag1_VK_chain:
(SEQ ID NO: 39)
MSVPTQVLGLLLLWLTDARCDIQLTQSPASLAVSLGQRATISCKASQSVDYDGDSYLNWYQQIPGQPP
KLLIYDASNLVSGIPPRFSGSGSGTDFTLNIHPVEKVDAATYHCQQSTEDPWTFGGGTKLEIKRTVAA
PSVFIFPPSDEQLKSGTASVVCLLNNFYPREAKVQWKVDNALQSGNSQESVTEQDSKDSTYSLSSTLT
LSKADYEKHKVYACEVTHQGLSSPVTKSFNRGECDKTHTCPPCPAPELLGGPSVFLFPPKPKDTLMIS
RTPEVTCVVVDVSHEDPEVKFNWYVDGVEVHNAKTKPREEQYSSTYRVVSVLTVLHQDWLNGKEYKCK
VSNKALPAPIEKTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK
Domain arrangement: Leader-VK-CK-Hinge-CH2*-CH3
CD19-F6-ICb_Frag2_VH_chain:
(SEQ ID NO: 40)
MEWSWVFLFFLSVTTGVHSQVQLQQSGAELVRPGSSVKISCKASGYAFSSYWMNWVKQRPGQGLEWIG
QIWPGDGDTNYNGKFKGKATLTADESSSTAYMQLSSLASEDSAVYFCARRETTTVGRYYYAMDYWGQG
TTVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQYSSTYRVVSVLTVLHQDWL
NGKEYKCKVSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPGSTGS
EVQLVQSGAEVKKSGESLKISCKGSGYSFTSYWIGWVRQMPGKGLEWMGIFYPGDSSTRYSPSFQGQV
TISADKSVNTAYLQWSSLKASDTAMYYCARRRNWGNAFDIWGQGTMVTVSSRTVAAPSVFIFPPSDEQ
LKSGTASVVCLLNNFYPREAKVQWKVDNALQSGNSQESVTEQDSKDSTYSLSSTLTLSKADYEKHKVY
ACEVTHQGLSSPVTKSFNRGEC
Domain arrangement: Leader-VH-CH1-Hinge-CH2*-CH3-STGS-Icb-VH-CK
CD19-F6-ICb_Frag3_Light_chain (identical to CD19-F5-ICb_Frag3_
Light_chain):
(SEQ ID NO: 41)
MSVPTQVLGLLLLWLTDARCEIVLTQSPGTLSLSPGERATLSCRASQSVSSSYLAWYQQKPGQAPRLL
IYGASSRATGIPDRFSGSGSGTDFTLTISRLEPEDFAVYYCQQYGSSTWTFGQGTKVEIKASTKGPSV
FPLAPSSKSTSGGTAALGCLVKDYFPEPVTVSWNSGALTSGVHTFPAVLQSSGLYSLSSVVTVPSSSL
GTQTYICNVNHKPSNTKVDKRVEPKSCDKTHS
Domain arrangement: Leader-Icb-VK-CH1

### Example 2

### Comparison of Monovalent Anti-CD19 Proteins and Convention IgG1 Antibodies in Cytotoxicity Assays

The anti-CD19 monovalent F5 protein of Example 1 was evaluated in a classic cytotoxicity assay using NK cells and tumor target cells. For comparison, a full-length antibody of human IgG1 isotype comprising the same CD19-binding VH and VL domains was also tested.

Briefly, the cytolytic activity of NK cells from Buffy coat from donors was assessed in a classic 4-h ⁵¹Cr-release assay in U-bottom 96 well plates. Daudi tumor cells (Burkitt's lymphoma; CD19-positive) were labelled with ⁵¹Cr, then mixed with NK cells at an effector/target ratio equal to 10:1 (25,000:2500), in the presence of test antibodies at dilution ranges starting from 10 μg/mL (or 100 μg/mL) with 1/10 dilution. Assays were in cRPMI, 150 μL final/well, in triplicates.

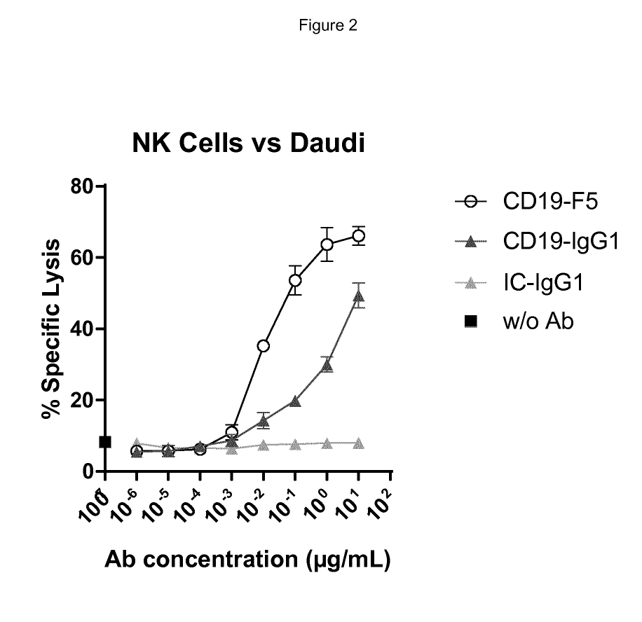

Results are shown in FIG. 2. The F5 protein as a monovalent, monospecific CD19 binding protein was surprisingly potent in its ability to mediate NK cell cytotoxicity of the tumor cells. The monovalent protein (F5) is superior to conventional anti-CD19 IgG1 antibody for recruiting NK cells against targets and inducing ADCC activity, despite the ability of the conventional anti-CD19 IgG1 to bind CD19 in bivalent manner (two antigen binding domains).

### Example 3

### In Vitro Potency of Monovalent CD20 Binding Proteins

New monovalent binding proteins were further constructed in an attempt to generate an agent that could improve on the most potent new generation of Fc enhanced antibodies. In these experiments as the comparison antibody we selected the commercial antibody GA101 (GAZYVA®, Gazyvaro®, obinutuzumab, Roche Pharmaceuticals), which is an Fc-modified human IgG1 antibody having enhanced CD16A binding as a result of hypofucosylated N-linked glycosylation.

A new set of heterotrimeric monovalent proteins were produced as a F5 protein, a F6 protein, and a F5+ protein having modified Fc domains to increase binding to human Fc domain, as well as a F6 protein. The proteins was made from the association of three polypeptide chains having the same domain arrangements and amino acid sequences as the proteins in Example 1, except that the anti-CD19 VH/VL pair were substituted by the respective VH and VL pair from the anti-CD20 antibody known as GA101 (GAZYVA®, Gazyvaro®, obinutuzumab, Roche Pharmaceuticals) and that in F5+ two amino acid substitutions were made in the CH2 domain of the heavy chain to increase binding affinity for human CD16A (referred to as “F5+”). Proteins were produced as in Example 1. The domain structures are shown in FIG. 1B.

Lysis of Daudi cells by CD20 monovalent and monospecific F5, F5+ and F6 antibodies were compared to the commercial antibody GA101 (GAZYVA®). Cytotoxicity assays were carried out as in Example 2. F5 and F5+ antibodies mediated ADCC toward Daudi cells, while F6 (lacking effector function) did not.

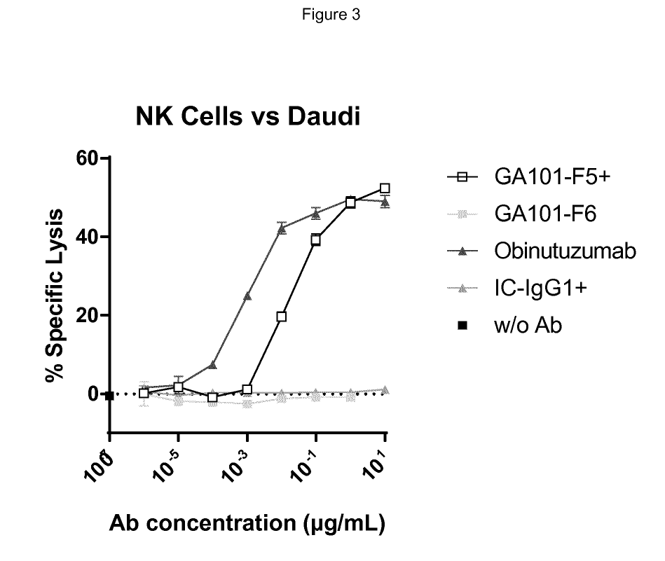

Obinutuzumab displayed the greatest potency in mediating cytotoxicity in this assay. Results are shown in FIG. 3 comparing GA101-F5+ monovalent protein to obinutuzumab (as a full-length bivalent binding antibody). Obinutuzumab was more potent in mediating cytotoxicity (approximately 10-fold improved EC₅₀) than the GA101-F5+ monovalent binding protein.

### Example 4

### In Vivo Potency of Monovalent CD20 Binding Proteins

To further investigate the monovalent anti-CD20 proteins, we tested the in vivo anti-tumor activity of the proteins of Example 3, again in comparison the commercial antibody GA101 (GAZYVA®, Gazyvaro®, obinutuzumab, Roche Pharmaceuticals).

CB17 SCID mice were engrafted with the CD20-expressing Raji tumor cell lines (5 million cells) and treated one day after engraftment with a single administration of 25 μg of either: (a) GA101-F5 monovalent protein of Example 3, (b) full length GA101 (obinutuzumab), or (c) GA101-F6 monovalent protein of Example 3 (which lacks effector function due to loss of binding to human Fcγ receptors).

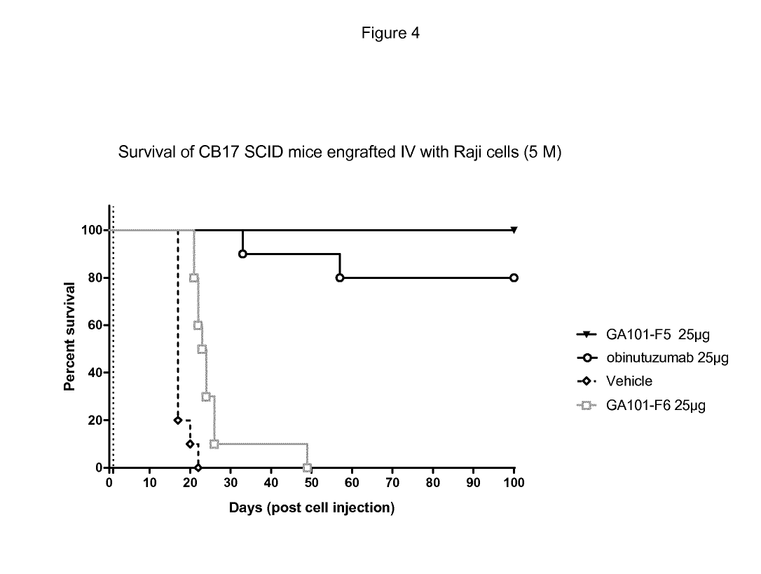

Results for the cytotoxicity assays are shown in FIG. 4. While the GA101-F6 monovalent protein did not significantly increase survival of mice, both the GA101-F5 monovalent protein and obinutuzumab demonstrated potent activity in enhancing survival of mice. Surprisingly, the GA101-F5 monovalent protein led to a greater survival benefit than obinutuzumab. Consequently, while obinutuzumab may display greater potency in in vitro cell cytotoxicity assays, the monovalent proteins as F5 proteins bearing a wild-type IgG1 Fc domain of anti-CD20 antibodies are superior to ADCC optimized anti-CD20 obinutuzumab abs for antitumor activity in vivo. These monovalent proteins appear to have advantageous properties that emerge in vivo; one possibility is that their reduced size (compared to a conventional IgG antibody) enhances tissue penetration (e.g. tumor tissue penetration) and thus efficacy in solid tumors. F5+ proteins having modified Fc domains are expected to provide further enhancement in anti-tumor potency in vivo. The monovalent proteins may thus have particular advantages in treating solid tumors that do not emerge when only in vitro assays were considered.

### Example 5

### Monovalent EGFR Binding Proteins

Monovalent binding proteins were further constructed in an attempt to generate an agent that could improve on the most widely used anti-EGFR antibody, cetuximab. Cetuximab (commercialized as Erbitux™, Merck KGaA, Bristol Myers Squibb, Eli Lilly) is a human IgG1 antibody that binds and inhibits human epidermal growth factor receptor (EGFR).

Monovalent heterotrimeric proteins were produced as a F5+ protein having modified Fc domains to increase binding to human Fc domain, as well as a F6 protein. The proteins was made from the association of three polypeptide chains having the same domain arrangements and amino acid sequences as the proteins in Example 1, except that the anti-CD19 VH/VL pair were substituted by the respective VH and VL pair from cetuximab and that in the F5+ protein two amino acid substitutions were made in the CH2 domain of the heavy chain to increase binding affinity for human CD16A. Proteins were produced as in Example 1. The domain structures of the three polypeptide chains of the proteins are shown in FIG. 1C.

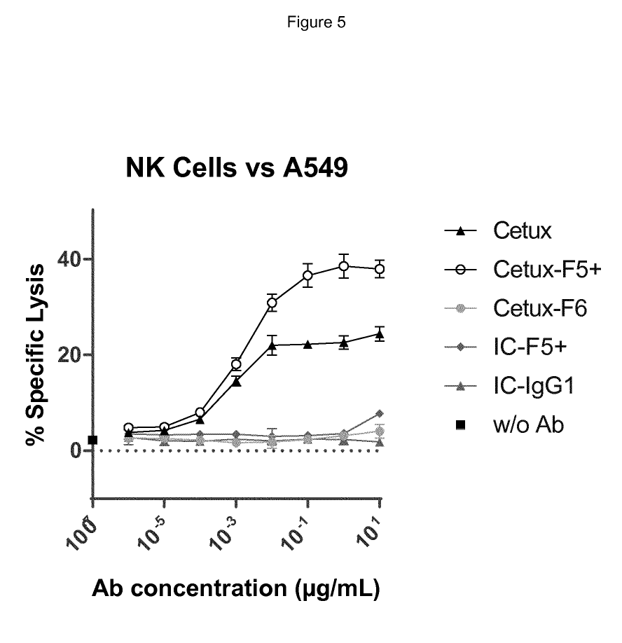

Lysis of A549 tumor cells by anti-EGFR monovalent and monospecific F5+ and F6 antibodies were compared to the commercial antibody cetuximab. Cytotoxicity assays were carried out as in Example 2. While neither F6 (Cetux-F6; lacking effector function), F5+ isotype control (IC-F5+) nor cetuximab isotype control (IC-IgG1) mediated any cytotoxicity towards the A549 tumor cells, both anti-EGFR F5+ proteins (Cetux-F5+) and cetuximab mediated potent cytotoxicity towards the A549 tumor cells. Results are shown in FIG. 5. The Cetux-F5+ protein was more potent in mediating cytotoxicity than cetuximab, despite the ability of cetuximab to bind its target in bivalent manner.

### Example 6

### Heterodimeric Monovalent Binding Proteins

Further monovalent proteins that bind CD19, CD20, EGFR or PD-L1 were designed as heterodimeric “M5” proteins. The domain structure of the dimeric M5 protein is shown in FIG. 6, wherein the interchain bonds between hinge domains (indicated between CH1/Cκ and CH2 domains on a chain) and interchain bonds between the CH1 and Cκ domains are interchain disulfide bonds. These proteins differ from the proteins of Examples 1-4 by the absence of protein domains (i.e. the ICb variable domains and CH1 or CK domains) positioned at the C-terminal end of the Fc domain.

New protein constructions with dimeric Fc domains were developed that share many of the advantages of the proteins of Examples 1-4 but lack any protein domains at the C-terminus of the Fc domain. These proteins may have advantages in vivo, particularly in solid tumors, due to further reduced size that may, for example in turn enhance tissue (or tumor tissue) penetration. The anti-CD19, CD20 and EGFR VH and VL variable domains used to construct these proteins were the same ones used in Examples 1-4. The anti-PD-L1 VH and VL variable domains were obtained from an anti-PD-L1 antibody that binds PD-L1 and inhibits the interaction between PD-L1 and its natural ligand PD-1.

The heterodimeric proteins comprised one polypeptide chain with a domain arrangement V_{H}-(CH1/Cκ)-CH2-CH3 and a second polypeptide chain with a domain arrangement Vκ-(CH1 or Cκ)-CH2-CH3, and wherein both chains lacked any variable domain or other protein domain (or amino acid sequence) fused to the CH3 domain. A hinge is placed between the CH1 domain and CH2 domain in one chain, and between the Cκ domain and the CH2 domain in the other chain. The two chains associate by CH1-Cκ dimerization to form disulfide linked dimers.

For each protein, a variant with wild-type Fc domain was produced (designated “M5”) as well as a variant having a substitution of two residues in the Fc domain that enhances binding to human FcγRIIIa (designated “M5+”).

Proteins are cloned, produced and purified as in Example 1. The amino acid sequences of the two polypeptide chains of the M5 and M5+ proteins are shown below, as well as their domain arrangement indicated below each amino acid sequence. The sequences shown include leader sequences (underlined); it will be appreciated that amino sequences can also be represented as mature sequences without the indicated leader sequences. The domains structures are shown in FIG. 6.

### Anti-CD19-M5 Protein:

CD19-M5_Frag1_VK_chain:
(SEQ ID NO: 42)
MSVPTQVLGLLLLWLTDARCDIQLTQSPASLAVSLGQRATISCKASQSVDYDGDSYLNWYQQIPGQPP
KLLIYDASNLVSGIPPRFSGSGSGTDFTLNIHPVEKVDAATYHCQQSTEDPWTFGGGTKLEIKRTVAA
PSVFIFPPSDEQLKSGTASVVCLLNNFYPREAKVQWKVDNALQSGNSQESVTEQDSKDSTYSLSSTLT
LSKADYEKHKVYACEVTHQGLSSPVTKSFNRGECDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK
Domain arrangement: Leader-VK-CK-Hinge-CH2-CH3
CD19-M5_Frag2_VH_chain:
(SEQ ID NO: 43)
MEWSWVFLFFLSVTTGVHSQVQLQQSGAELVRPGSSVKISCKASGYAFSSYWMNWVKQRPGQGLEWIG
QIWPGDGDTNYNGKFKGKATLTADESSSTAYMQLSSLASEDSAVYFCARRETTTVGRYYYAMDYWGQG
TTVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQYSSTYRVVSVLTVLHQDWL
NGKEYKCKVSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPGK
Domain arrangement: Leader-VH-CH1-Hinge-CH2-CH3

### Anti-CD20-M5 Protein: (Obinutuzumab)

CD20-M5_Frag1_VK_chain:
(SEQ ID NO: 44)
MSVPTQVLGLLLLWLTDARCDIVMTQTPLSLPVTPGEPASISCRSSKSLLHSNGITYLYWYLQKPGQS
PQLLIYQMSNLVSGVPDRFSGSGSGTDFTLKISRVEAEDVGVYYCAQNLELPYTFGGGTKVEIKRTVA
APSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK
Domain arrangement: Leader-VK-CK-Hinge-CH2-CH3
CD20-M5_Frag2_VH_chain:
(SEQ ID NO: 45)
MEWSWVFLFFLSVTTGVHSQVQLVQSGAEVKKPGSSVKVSCKASGYAFSYSWINWVRQAPGQGLEWMG
RIFPGDGDTDYNGKFKGRVTITADKSTSTAYMELSSLRSEDTAVYYCARNVFDGYWLVYWGQGTLVTV
SSASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYSSTYRVVSVLTVLHQDWLNGKEY
KCKVSNKALPAPIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLSPGK
Domain arrangement: Leader-VH-CH1-Hinge-CH2-CH3

### Anti-EGFR-M5 Protein (Cetuximab):

Cetux-M5_Frag1_VK_chain:
(SEQ ID NO: 46)
MSVPTQVLGLLLLWLTDARCDILLTQSPVILSVSPGERVSFSCRASQSIGTNIHWYQQRTNGSPRLLI
KYASESISGIPSRFSGSGSGTDFTLSINSVESEDIADYYCQQNNNWPTTFGAGTKLELRTVAAPSVFI
FPPSDEQLKSGTASVVCLLNNFYPREAKVQWKVDNALQSGNSQESVTEQDSKDSTYSLSSTLTLSKAD
YEKHKVYACEVTHQGLSSPVTKSFNRGECDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK
Domain arrangement: Leader-VK-CK-Hinge-CH2-CH3
Cetux-M5_Frag2_VH_chain:
(SEQ ID NO: 47)
MEWSWVFLFFLSVTTGVHSQVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARALTYYDYEFAYWGQGTLVTV
SAASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYSSTYRVVSVLTVLHQDWLNGKEY
KCKVSNKALPAPIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLSPGK
Domain arrangement: Leader-VH-CH1-Hinge-CH2-CH3

### Anti-PD-L1-M5 Protein

Anti-PD-L1 BMS-936559/MDX-1105
PD-L1-M5_Frag1_VK_chain:
(SEQ ID NO: 48)
MSVPTQVLGLLLLWLTDARCEIVLTQSPATLSLSPGERATLSCRASQSVSSYLAWYQQKPGQAPRLLI
YDASNRATGIPARFSGSGSGTDFTLTISSLEPEDFAVYYCQQRSNWPTFGQGTKVEIKRTVAAPSVFI
FPPSDEQLKSGTASVVCLLNNFYPREAKVQWKVDNALQSGNSQESVTEQDSKDSTYSLSSTLTLSKAD
YEKHKVYACEVTHQGLSSPVTKSFNRGECDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK
Domain arrangement: Leader-VK-CK-Hinge-CH2-CH3
PD-L1-M5_Frag2_VH_chain:
(SEQ ID NO: 49)
MEWSWVFLFFLSVTTGVHSQVQLVQSGAEVKKPGSSVKVSCKTSGDTFSTYAISWVRQAPGQGLEWMG
GIIPIFGKAHYAQKFQGRVTITADESTSTAYMELSSLRSEDTAVYFCARKFHFVSGSPFGMDVWGQGT
TVTVSSASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYSSTYRVVSVLTVLHQDWLN
GKEYKCKVSNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSLSLSPGK
Domain arrangement: Leader-VH-CH1-Hinge-CH2-CH3

### Anti-CD19-M5+ Protein:

CD19-M5+_Frag1_VK_chain:
(SEQ ID NO: 50)
MSVPTQVLGLLLLWLTDARCDIQLTQSPASLAVSLGQRATISCKASQSVDYDGDSYLNWYQQIPGQPP
KLLIYDASNLVSGIPPRFSGSGSGTDFTLNIHPVEKVDAATYHCQQSTEDPWTFGGGTKLEIKRTVAA
PSVFIFPPSDEQLKSGTASVVCLLNNFYPREAKVQWKVDNALQSGNSQESVTEQDSKDSTYSLSSTLT
LSKADYEKHKVYACEVTHQGLSSPVTKSFNRGECDKTHTCPPCPAPELLGGPDVFLFPPKPKDTLMIS
RTPEVTCVVVDVSHEDPEVKFNWYVDGVEVHNAKTKPREEQYNSTYRVVSVLTVLHQDWLNGKEYKCK
VSNKALPAPEEKTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK
Domain arrangement: Leader-VK-CK-Hinge-CH2-CH3+
CD19-M5+_Frag2_VH_chain:
(SEQ ID NO: 51)
MEWSWVFLFFLSVTTGVHSQVQLQQSGAELVRPGSSVKISCKASGYAFSSYWMNWVKQRPGQGLEWIG
QIWPGDGDTNYNGKFKGKATLTADESSSTAYMQLSSLASEDSAVYFCARRETTTVGRYYYAMDYWGQG
TTVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPELLGGPDVFLFPPK
PKDTLMISRTPEVTCVVVDVSHEDPEVKFNWYVDGVEVHNAKTKPREEQYNSTYRVVSVLTVLHQDWL
NGKEYKCKVSNKALPAPEEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPGK
Domain arrangement: Leader-VH-CH1-Hinge-CH2-CH3+

### Anti-CD20-M5+ Protein: (Obinutuzumab)

CD20-M5+_Frag1_VK_chain:
(SEQ ID NO: 52)
MSVPTQVLGLLLLWLTDARCDIVMTQTPLSLPVTPGEPASISCRSSKSLLHSNGITYLYWYLQKPGQS
PQLLIYQMSNLVSGVPDRFSGSGSGTDFTLKISRVEAEDVGVYYCAQNLELPYTFGGGTKVEIKRTVA
APSVFIFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGECDKTHTCPPCPAPELLGGPDVFLFPPKPKDTLMI
SRTPEVTCVVVDVSHEDPEVKFNWYVDGVEVHNAKTKPREEQYNSTYRVVSVLTVLHQDWLNGKEYKC
KVSNKALPAPEEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK
Domain arrangement: Leader-VK-CK-Hinge-CH2-CH3+
CD20-M5+_Frag2_VH_chain:
(SEQ ID NO: 53)
MEWSWVFLFFLSVTTGVHSQVQLVQSGAEVKKPGSSVKVSCKASGYAFSYSWINWVRQAPGQGLEWMG
RIFPGDGDTDYNGKFKGRVTITADKSTSTAYMELSSLRSEDTAVYYCARNVFDGYWLVYWGQGTLVTV
SSASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAPELLGGPDVFLFPPKPKDTL
MISRTPEVTCVVVDVSHEDPEVKFNWYVDGVEVHNAKTKPREEQYNSTYRVVSVLTVLHQDWLNGKEY
KCKVSNKALPAPEEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLSPGK
Domain arrangement: Leader-VH-CH1-Hinge-CH2-CH3+

### Anti-EGFR-M5+ Protein (Cetuximab):

Cetux-M5+_Frag1_VK_chain:
(SEQ ID NO: 54)
MSVPTQVLGLLLLWLTDARCDILLTQSPVILSVSPGERVSFSCRASQSIGTNIHWYQQRTNGSPRLLI
KYASESISGIPSRFSGSGSGTDFTLSINSVESEDIADYYCQQNNNWPTTFGAGTKLELRTVAAPSVFI
FPPSDEQLKSGTASVVCLLNNFYPREAKVQWKVDNALQSGNSQESVTEQDSKDSTYSLSSTLTLSKAD
YEKHKVYACEVTHQGLSSPVTKSFNRGECDKTHTCPPCPAPELLGGPDVFLFPPKPKDTLMISRTPEV
TCVVVDVSHEDPEVKFNWYVDGVEVHNAKTKPREEQYNSTYRVVSVLTVLHQDWLNGKEYKCKVSNKA
LPAPEEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK
Domain arrangement: Leader-VK-CK-Hinge-CH2-CH3+
Cetux-M5+_Frag2_VH_chain:
(SEQ ID NO: 55)
MEWSWVFLFFLSVTTGVHSQVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARALTYYDYEFAYWGQGTLVTV
SAASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKRVEPKSCDKTHTCPPCPAPELLGGPDVFLFPPKPKDTL
MISRTPEVTCVVVDVSHEDPEVKFNWYVDGVEVHNAKTKPREEQYNSTYRVVSVLTVLHQDWLNGKEY
KCKVSNKALPAPEEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLSPGK
Domain arrangement: Leader-VH-CH1-Hinge-CH2-CH3+

### Anti-PD-L1-M5+ Protein

Anti-PD-L1 BMS-936559/MDX-1105
PD-L1-M5+_Frag1_VK_chain:
(SEQ ID NO: 56)
MSVPTQVLGLLLLWLTDARCEIVLTQSPATLSLSPGERATLSCRASQSVSSYLAWYQQKPGQAPRLLI
YDASNRATGIPARFSGSGSGTDFTLTISSLEPEDFAVYYCQQRSNWPTFGQGTKVEIKRTVAAPSVFI
FPPSDEQLKSGTASVVCLLNNFYPREAKVQWKVDNALQSGNSQESVTEQDSKDSTYSLSSTLTLSKAD
YEKHKVYACEVTHQGLSSPVTKSFNRGECDKTHTCPPCPAPELLGGPDVFLFPPKPKDTLMISRTPEV
TCVVVDVSHEDPEVKFNWYVDGVEVHNAKTKPREEQYNSTYRVVSVLTVLHQDWLNGKEYKCKVSNKA
LPAPEEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK
Domain arrangement: Leader-VK-CK-Hinge-CH2-CH3+
PD-L1-M5+_Frag2_VH_chain:
(SEQ ID NO: 57)
MEWSWVFLFFLSVTTGVHSQVQLVQSGAEVKKPGSSVKVSCKTSGDTFSTYAISWVRQAPGQGLEWMG
GIIPIFGKAHYAQKFQGRVTITADESTSTAYMELSSLRSEDTAVYFCARKFHFVSGSPFGMDVWGQGT
TVTVSSASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELLGGPDVFLFPPKP
KDTLMISRTPEVTCVVVDVSHEDPEVKFNWYVDGVEVHNAKTKPREEQYNSTYRVVSVLTVLHQDWLN
GKEYKCKVSNKALPAPEEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSLSLSPGK
Domain arrangement: Leader-VH-CH1-Hinge-CH2-CH3

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e. g., all exact exemplary values provided with respect to a particular factor or measurement can be considered to also provide a corresponding approximate measurement, modified by “about,” where appropriate). All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as reference to an element or elements is intended to provide support for a similar aspect or embodiment of the invention that “consists of,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1-35. (canceled)

36. A heterodimeric antigen binding protein having a single VH-VL domain pair, the protein comprising:
a first polypeptide chain comprising an immunoglobulin variable domain fused to a CK domain in turn fused at its C-terminus to a human Fc domain, and
a second polypeptide chain comprising an immunoglobulin variable domain fused to a CH1 domain in turn fused at its C-terminus to a human Fc domain,
wherein one of the variable domains is a heavy chain variable domain and the other is a light chain variable domain, wherein the V-Cκ unit of the first chain is bound, by CH1-Cκ dimerization, to the V-CH1 unit of the second chain such that the variable domain of the first chain and the second chain form an antigen binding domain, and a dimeric Fc domain capable of binding to a human CD16 polypeptide is formed.

37. The protein of claim 36, wherein the variable domain of the first polypeptide chain is a VH domain and the variable domain of the second polypeptide chain is a VL domain.

38. The protein of claim 36, wherein the variable domain of the first polypeptide chain is a VL domain and the variable domain of the second polypeptide chain is a VH domain.

39. The protein of claim 36, wherein the first and second polypeptide chain are free of further immunoglobulin variable domains.

40. The protein of claim 36, wherein the protein comprises a single VH domain and a single VL domain.

41. The protein of claim 36, wherein the first and second polypeptide chains are free of any protein domains fused to the C-terminus of the Fc domain.

42. The protein of claim 36, wherein said antigen binding domain binds to an antigen of interest expressed at the surface of a cell to be depleted.

43. The protein of claim 36, wherein the protein mediates ADCC toward the target cell.

44. The protein of claim 36, wherein the protein is a heterodimer with a dimeric Fc domain, having the domain arrangement:
V-CK-Fc domain
V-CH1-Fc domain
wherein one V is a light chain variable domain and the other V is a heavy chain variable domain, wherein the V pair form antigen binding domain that binds an antigen of interest.

45. The protein of claim 36, wherein the protein is a heterodimer with a dimeric Fc domain, having the domain arrangement:
V-CK-hinge-Fc domain
V-CH1-hinge-Fc domain
wherein one V is a light chain variable domain and the other V is a heavy chain variable domain, wherein the V pair form an antigen binding domain that binds an antigen of interest.

46. The protein of claim 36, wherein the monovalent protein is a heterodimer with a dimeric Fc domain, having the domain arrangement:
V-linker-CK-hinge-Fe domain
V-linker-CH1-hinge-Fc domain
wherein one V is a light chain variable domain and the other V is a heavy chain variable domain, wherein the V pair form an antigen binding domain that binds an antigen of interest.

47. The protein of claim 36, wherein the two polypeptide chains are bound by non-covalent bonds between complementary VH and VL domains and by non-covalent bonds between complementary CH1 and Cκ domains.

48. The protein of claim 36, wherein the two polypeptide chains are bound by non-covalent bonds between CH3 domains of the respective Fe domains.

49. The protein of claim 36, wherein the two polypeptide chains are bound by disulfide bonding between complementary CH1 and Cκ domains.

50. The protein of claim 36, wherein the two polypeptide chains are bound by disulfide bonds between complementary hinge domains.

51. The protein of claim 36, wherein the Fc domain(s) comprises N-linked glycosylation at residue 297 (Kabat EU numbering).

52. The protein of claim 36, wherein the protein comprises: (a) a first polypeptide chain comprising a variable domain fused at its C-terminus to a polypeptide chain comprising an amino acid sequence at least 60%, 70%, 80%, 90%, 95% or 99% identical to the sequence shown in SEQ ID NO: 12, and (b) a second polypeptide chain comprising a variable domain fused at its C-terminus to a polypeptide chain comprising an amino acid sequence at least 60%, 70%, 80%, 90%, 95% or 99% identical to the sequence shown in SEQ ID NO: 13, wherein one of the variable domains is a VH domain and the other is a VL domain.

53. The protein of claim 36, wherein the protein binds human PD-L1 and inhibits the interaction of PD-L1 with PD-1.

54. The protein of claim 36, wherein the antigen binding domain binds an antigen present on a target cell present in tumor tissue or tumor-adjacent tissue, wherein the antigen is CD19, CD20 or EGFR.

55. A pharmaceutical composition comprising a protein of claim 36, and a pharmaceutically acceptable carrier.

56. A method of treating a cancer in a subject comprising administering to the subject a composition of claim 55.

57. A method of making a heterodimeric or heterotrimeric protein, comprising:
(a) providing a first nucleic acid encoding a first polypeptide chain of a protein of claim 36;
(b) providing a second nucleic acid encoding a second polypeptide chain of a protein of claim 36; and
(c) expressing said first and second (and optionally a third) nucleic acids in a host cell to produce a protein comprising said first and second (and optionally third) polypeptide chains, respectively; loading the protein produced onto an affinity purification support, optionally a Protein-A support, and recovering a heterodimeric (or heterotrimeric) protein.


SEQUENCE LISTING (59 sequences)

SEQ ID NO: 1 - protein, 118 aa, Artificial [sequence omitted]

SEQ ID NO: 2 - protein, 107 aa, Artificial [sequence omitted]

SEQ ID NO: 3 - protein, 123 aa, Artificial
  Chimeric
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 4 - protein, 106 aa, Artificial
  Chimeric
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 5 - protein, 121 aa, Artificial
  Chimeric
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 6 - protein, 108 aa, Artificial
  Chimeric
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 7 - protein, 120 aa, Artificial
  Chimeric
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 8 - protein, 110 aa, Artificial
  Chimeric
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 9 - protein, 13 aa, homo sapiens
THTCSSCPAPELL

SEQ ID NO: 10 - protein, 15 aa, homo sapiens
NASSMCVPSPAPELL

SEQ ID NO: 11 - protein, 13 aa, homo sapiens
THTSPPSPAPELL

SEQ ID NO: 12 - protein, 334 aa, Artificial
  Human derived construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 330 aa, Artificial
  Homo sapiens derived construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 334 aa, Artificial
  homo sapiens derived construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 330 aa, Artificial
  Homo sapiens based construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 445 aa, Artificial
  Homo sapiens based construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 454 aa, Artificial
  Homo sapiens based construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 446 aa, Artificial
  Homo sapiens based construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 449 aa, Artificial
  Homo sapiens based construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 440 aa, Artificial
  Homo sapiens based construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 448 aa, Artificial
  Homo sapiens based construct
VQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 440 aa, Artificial
  Homo sapiens based construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 453 aa, Artificial
  Homo sapiens based construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 445 aa, Artificial
  Homo sapiens based construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 454 aa, Artificial
  Homo sapiens based construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 446 aa, Artificial
  Homo sapiens based construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 449 aa, Artificial
  Homo sapiens based construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 440 aa, Artificial
  Homo sapiens based construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 449 aa, Artificial
  Homo sapiens based construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 440 aa, Artificial
  Homo sapiens based construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 453 aa, Artificial
  Homo sapiens based construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 333 bp, Artificial
  Chimeric
gacattcagctgacccaatctccagcttctttggctgtgtctctagggcagagggccaccatctcctgcaaggccagccaaagtgttgattatgatggtgatagttatttgaactggtaccaacagataccaggacagccacccaaactcctcatctatgatgcatccaatctagtatctgggattccacccaggtttagtggcagtgggtctgggacagacttcaccctcaacatccatcctgtggagaaggtggatgctgcaacctatcactgtcagcaaagtactgaggacccttggacgttcggtggaggcaccaagctggaaatcaaa

SEQ ID NO: 33 - protein, 111 aa, Artificial
  Chimeric
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 34 - DNA, 372 bp, Artificial
  Chimeric
caggttcagctgcagcagtctggggctgagctggtgcggcctgggtcctcagtgaagatttcctgcaaagcatctggctacgcattcagtagctactggatgaactgggtgaagcagaggcctggacagggtcttgagtggattggacagatttggcctggagatggtgatactaactacaacggaaagttcaagggcaaggccacactgactgcagacgaatcctccagcacagcctacatgcagctcagcagcctggcctctgaggactctgcggtctatttctgtgcaagacgagaaacgaccactgtcgggcgttattactatgctatggactactggggtcaaggaaccacagtcaccgtctcctca

SEQ ID NO: 35 - protein, 124 aa, Artificial
  Chimeric
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 36 - protein, 465 aa, Artificial
  Homo sapiens based construct
MSVPTQVLGLLLLWLTDARCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 702 aa, Artificial
  Homo sapiens based construct
MEWSWVFLFFLSVTTGVHSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLVQSGAEVKKSGESLKISCKGSGYSFTSYWIGWVRQMPGKGLEWMGIFYPGDSSTRYSPSFQGQVTISADKSVNTAYLQWSSLKASDTAMYYCARRRNWGNAFDIWGQGTMVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 236 aa, Artificial
  Homo sapiens based construct
MSVPTQVLGLLLLWLTDARCEIVLTQSPGTLSLSPGERATLSCRASQSVSSSYLAWYQQKPGQAPRLLIYGASSRATGIPDRFSGSGSGTDFTLTISRLEPEDFAVYYCQQYGSSTWTFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 39 - protein, 465 aa, Artificial
  Homo sapiens based construct
MSVPTQVLGLLLLWLTDARCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 702 aa, Artificial
  Homo sapiens based construct
MEWSWVFLFFLSVTTGVHSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLVQSGAEVKKSGESLKISCKGSGYSFTSYWIGWVRQMPGKGLEWMGIFYPGDSSTRYSPSFQGQVTISADKSVNTAYLQWSSLKASDTAMYYCARRRNWGNAFDIWGQGTMVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 236 aa, Artificial; identical to SEQ ID NO: 38
  Homo sapiens based construct

SEQ ID NO: 42 - protein, 465 aa, Artificial; identical to SEQ ID NO: 36
  Homo sapiens based construct

SEQ ID NO: 43 - protein, 473 aa, Artificial
  Homo sapiens based construct
MEWSWVFLFFLSVTTGVHSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 466 aa, Artificial
  Homo sapiens based construct
MSVPTQVLGLLLLWLTDARCDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 468 aa, Artificial
  Homo sapiens based construct
MEWSWVFLFFLSVTTGVHSQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 460 aa, Artificial
  Homo sapiens based construct
MSVPTQVLGLLLLWLTDARCDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 468 aa, Artificial
  Homo sapiens based construct
MEWSWVFLFFLSVTTGVHSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 460 aa, Artificial
  Homo sapiens based construct
MSVPTQVLGLLLLWLTDARCEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 472 aa, Artificial
  Homo sapiens based construct
MEWSWVFLFFLSVTTGVHSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 465 aa, Artificial
  Homo sapiens based construct
MSVPTQVLGLLLLWLTDARCDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 473 aa, Artificial
  Homo sapiens based construct
MEWSWVFLFFLSVTTGVHSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 466 aa, Artificial
  Homo sapiens based construct
MSVPTQVLGLLLLWLTDARCDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 468 aa, Artificial
  Homo sapiens based construct
MEWSWVFLFFLSVTTGVHSQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 460 aa, Artificial
  Homo sapiens based construct
MSVPTQVLGLLLLWLTDARCDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 468 aa, Artificial
  Homo sapiens based construct
MEWSWVFLFFLSVTTGVHSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 460 aa, Artificial
  Homo sapiens based construct
MSVPTQVLGLLLLWLTDARCEIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 472 aa, Artificial
  Homo sapiens based construct
MEWSWVFLFFLSVTTGVHSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 288 aa, Homo sapiens
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 59 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET